(19) United States
(12) Patent Application Publication
(10) Pub. No.: US 2006/0099709 A1
(43) Pub. Date: May 11, 2006

(54) Targeted tumor therapy by use of recombinant adenovirus vectors that selectively replicate in hypoxic regions of tumors

(76) Inventors: Chuan-Yuan Li, Durham, NC (US); Qian Huang, Durham, NC (US); Mark W. Dewhirst, Durham, NC (US)

Correspondence Address: JENKINS, WILSON & TAYLOR, P. A., 3100 TOWER BLVD, SUITE 1200, DURHAM, NC, 27707, US

(21) Appl. No.: 10/529,071

(22) PCT Filed: Oct. 1, 2003
(86) PCT No.: PCT/US03/31097

§ 371 Date: Sep. 30, 2005

Related U.S. Application Data
(60) Provisional application No. 60/415,319, filed on Oct. 1, 2002.

(51) Int. Cl. C12N 15/861; A61K 48/00

(52) U.S. Cl. 435/456; 424/93.2

(57) ABSTRACT

The presently claimed subject matter provides conditionally replication competent adenoviral vectors that confer selective cytotoxicity on cells expressing HIF-1 by infecting cells that allow HIF-1 inducible promoters present within the vectors to function. Also provided are compositions and host cells based upon the vectors, as well as methods of propagating and using the vectors. The presently claimed subject matter further provides a method of inhibiting tumor growth by co-infecting cells in a tumor with a conditionally replication competent adenovirus vector in conjunction with a replication deficient adenovirus vector.

### CROSS-REFERENCE TO RELATED APPLICATION

This application is based on and claims priority to U.S. Provisional Patent Application Ser. No. 60/415,319, filed Oct. 1, 2002, herein incorporated by reference in its entirety.

### GRANT STATEMENT

This work was supported by grant CA81512 from the U.S. National Institute of Health (NIH). Thus, the U.S. government has certain rights in the presently claimed subject matter.

### TECHNICAL FIELD

The presently claimed subject matter generally relates to methods for propagating a conditionally replication competent adenovirus vector in a hypoxic cell. More particularly, the methods involve infecting hypoxic cells, for example a hypoxic cell in a tumor, with a conditionally replication competent adenovirus vector such that the adenovirus vector replicates in the hypoxic cell, killing the cell.

### TABLE OF ABBREVIATIONS

Ad adenovirus

AdCMV-EGFP adenovirus vector with the EGFP gene under transcriptional control of a constitutive CMV promoter

AdCMV-dsRed2 replication deficient adenovirus vector with the dsRed2 gene under transcriptional control of a constitutive CMV promoter

AdHRP-E1A-dsRed2 conditionally replication competent Ad vector with the Ad E1A gene under control of an HRP. Also constitutively expressed a red fluorescent marker

AdHRP-E1A-TNF-α conditionally replication competent adenovirus vector with the Ad E1A gene under control of an HRP and the TNFα gene under control of a constitutive CMV promoter

AdHRP-E1AE4-dsRed2 conditionally replication competent Ad vector with the Ad E1A and E4 genes under control of an HRP. Also constitutively expressed a red fluorescent marker

AdHRP-E4-dsRed2 conditionally replication competent Ad vector with the Ad E4 gene under control of an HRP. Also constitutively expressed a red fluorescent marker

ARNT aryl receptor nuclear translocator

CMV cytomegalovirus

DNAse I deoxyribonuclease I

DHFR dihydrofolate reductase

dsRed2 Discosoma sp. red fluorescent protein

E1A adenovirus early gene 1A

E1B adenovirus early gene 1B

E2A adenovirus early gene 2A

E2B adenovirus early gene 2B

E3 adenovirus early gene 3

E4 adenovirus early gene 4

EGFP enhanced green fluorescent protein

ex-Flk1 extracellular domain of an Flk1 receptor

HIF hypoxia inducible factor

HPRT hypoxanthine phosphoribosyl transferase

HRE hypoxia responsive element

HRP hypoxia responsive promoter

HRP-EGFP plasmid vector wherein the expression of EGFP is regulated by an HRP

hsp heat shock protein

HSV-tk herpes simplex virus thymidine kinase

IL2 interleukin 2

IL12 interleukin 12

kb kilobase

MOI multiplicity of infection

NIH National Institutes of Health

pfu plaque forming units

PGK phosphoglycerate kinase

PSA prostate specific antigen

pVHL von Hippel-Lindau protein

s-Flt1 a soluble form of the Flt1 receptor

SV40 simian virus 40

TAFs transcription-associated factors

Tm melting temperature

TNF-α tumor necrosis factor-alpha

TRE transcriptional regulatory element

VEGF vascular endothelial growth factor

VHL von Hippel-Lindau

### BACKGROUND ART

Despite significant advances in medical research and technology, cancer continues to be one of the leading causes of death in the United States and throughout the world. There are in excess of one million new cases of cancer reported in the United States alone, and more than half a million people die in this country every year from cancer.

Current treatments for cancer include surgical removal or radiation treatment of tumors, yet each has its limitations. In the former case, once a tumor has metastasized by invading the surrounding tissue or by moving to a distant site, it can be virtually impossible for the surgeon to remove all cancerous cells. Any such cells left behind can continue growing, leading to a recurrence of cancer following surgery. Current radiation therapy strategies are also frequently unsuccessful at curing a patient's cancer. Following radiation therapy, cancer can recur because it is often not possible to deliver a sufficiently high dose of radiation to kill all the tumor cells without at the same time injuring the surrounding normal tissue. Cancer can also recur because tumors show widely varying susceptibilities to radiation induced cell death. Thus, the inability of current treatment protocols to eliminate tumor cells is an important clinical limitation leading to unsuccessful cancer therapy (Lindegaard et al., 1996; Suit, 1996; Valter et al., 1999).

Newer treatment strategies are needed to address the challenges that result from the inability to successfully treat neoplastic disease. One of the major challenges facing the medical oncologist is selectivity: the ability to kill tumor cells without causing damage to normal cells in the surrounding area. Various current approaches take advantage of the fact that in most cases tumor cells grow more quickly than normal cells, so strategies designed to kill rapidly growing cells are somewhat selective for tumor cells (see Yazawa et al., 2002). However, these methods also kill certain cell types in the body that normally divide rapidly, most notably cells in the bone marrow, resulting in complications such as anemia and neutropenia (reviewed in Vose & Armitage, 1995). Other strategies are based upon the production of antibodies directed against tumor-specific antigens (reviewed in Sinkovics & Horvath, 2000), although few such antigens have been identified, limiting the applicability of these approaches. Thus, there is a need for new methods to enhance the selectivity of cancer treatment approaches.

Recently, attempts have been made to develop and use replication competent viruses that can selectively replicate in, and thereby kill, tumor cells (see e.g., Galanis et al., 2001). In this approach, viral vectors are genetically engineered to replicate specifically in targeted tumor cells. Successfully targeted tumor cells are then killed by virus-mediated cell lysis, which can lead to subsequent infection and killing of neighboring cells (Galanis et al., 2001).

The challenge presented by this approach is to find mechanisms that will allow the viruses to selectively target and/or replicate in tumor cells. To this point, selective replication schemes have been attempted that are based on specific genetic traits of tumor cells (see Galanis et al., 2001) and references therein). For example, one of the approaches to achieve tumor-specific virus replication involves the recombinant oncolytic adenovirus vector dl1520, or Onyx-015. Onyx-015 vectors have been designed in an attempt to provide selective replication in cells that have lost the p53 tumor suppressor gene (Bischoff et al., 1996; Ries & Kom, 2002). The design of Onyx-015 was based on the fact that successful adenovirus replication requires the inactivation of the cellular p53 protein, which is accomplished by the adenovirus E1B protein. Onyx-015 has a mutation in the E1B gene that destroys this p53-inactivation capability. The E1B mutation allows the virus to replicate in cells that lack p53 function, but prevents replication in cells with wild type p53. As p53 function is lost in over 50% of all tumors including about 70% of some cancers such as colorectal cancer (see e.g., Beroud & Soussi, 1998; Colman et al., 2000; Hickman et al., 2002), Onyx-15 can in theory be used for the treatment of more than half of all tumors. Unfortunately, recent controversies have developed regarding the specificity of Onyx-015 (see Goodrum & Ornelles, 1998; Rothmann et al., 1998; Dix et al., 2001; Ries & Kom, 2002). Some studies indicate that Onyx-015 can replicate even in tumor cells with wild-type p53 function (Goodrum & Ornelles, 1998; Rothmann et al., 1998). While this apparent contrast could possibly be reconciled by the fact that most tumor cells with normal p53 functions have defects in other parts of the p53 pathway, it nonetheless presents a limitation to the widespread use of this vector. Even so, there remains a need for a strategy for use in tumor cells that maintain wild type p53 function.

Another strategy for targeting the replication of adenovirus vectors to tumor cells involves the use of tumor- and/or tissue-specific promoters to control the expression of genes required for viral replication (reviewed in (Haviv & Curiel, 2001). A typical example is CN706 (Calydon, Inc., Sunnyvale, Calif., United States of America), in which the prostate-specific antigen (PSA) gene promoter drives the expression of the adenovirus EIA gene. See U.S. Pat. No. 5,871,726 to Henderson and Schuur. Specificity was also seen in another virus CV787, where the rat prostate-specific probasin promoter drives the expression of E1A while the PSA promoter drives the expression of E1B (Yu et al., 1999). Another attempt at this strategy involved the use of a MUC1 promoter to control the expression of E1A (Kurihara et al., 2000). The key for these types of strategies is the specificity of the promoter. Unfortunately, very few promoters have been identified that exhibit sufficient specificity to be useful in an anti-tumor strategy.

Thus, there exists a long-felt and continuing need in the art for effective therapies to specifically target and kill tumor cells in a subject. The presently claimed subject matter addresses this and other needs in the art.

### SUMMARY

The presently claimed subject matter provides an adenovirus vector comprising an adenovirus gene under the transcriptional control of a transcriptional regulatory element (TRE) comprising a minimal promoter and a hypoxia responsive element (HRE). In one embodiment, the adenovirus gene is selected from the group consisting of the E1A gene, the E1B gene, the E2A gene, the E2B gene, and the E4 gene. In one embodiment, the adenovirus vector comprises a second adenovirus gene under the transcriptional control of a transcriptional regulatory element (TRE). In one embodiment, the minimal promoter is selected from the group consisting of cytomegalovirus (CMV) minimal promoter, the human p-actin minimal promoter, the human EF2 minimal promoter, and the adenovirus E1B minimal promoter. In another embodiment, the CMV minimal promoter comprises SEQ ID NO: 1. In one embodiment, the hypoxia responsive element (HRE) is derived from the human vascular endothelial growth factor (VEGF) gene. In another embodiment, the HRE comprises five tandem copies of SEQ ID NO: 2. In one embodiment, the adenovirus vector further comprises a transgene. In one example, the transgene comprises a second adenovirus gene. In another example, the transgene comprises a nucleic acid encoding an immunostimulatory molecule. In yet another example, the transgene comprises a suicide gene.

The presently claimed subject matter also provides a composition comprising an adenovirus gene under the transcriptional control of a TRE comprising a minimal promoter and an HRE. In one example, the composition further comprises a pharmaceutically acceptable carrier.

The presently claimed subject matter also provides a method for suppressing tumor growth, the method comprising contacting a hypoxic cell in a tumor with an adenovirus vector, whereby the vector enters the cell and inhibits tumor growth. In one embodiment, the contacting is a result of intratumoral administration of the vector. In another embodiment, the contacting is a result of intravenous administration of the vector.

The presently claimed subject matter also provides a host cell comprising an adenovirus gene under the transcriptional control of a TRE comprising a minimal promoter and an HRE.

The presently claimed subject matter also provides a method for propagating an adenovirus specific for a hypoxic cell, the method comprising contacting a hypoxic cell with an adenovirus vector whereby the adenovirus is propagated to a titer of at least 10⁴ virus particles/cell.

The presently claimed subject matter also provides a method for conferring selective cytotoxicity on a target cell, the method comprising contacting a cell that allows an HRE to function with an adenovirus vector comprising an HRE, whereby the adenovirus vector enters the cell.

The presently claimed subject matter also provides a method of inhibiting tumor growth, the method comprising (a) contacting a hypoxic cell in a tumor with a first adenovirus vector, whereby the first adenovirus vector enters the cell, and (b) contacting the hypoxic cell with a replication deficient adenovirus vector, whereby the replication deficient adenovirus vector enters the cell. In one embodiment, the first adenovirus vector comprises an adenovirus gene under the transcriptional control of a TRE comprising an HRE. In another embodiment, the replication deficient adenovirus vector comprises a second gene under the transcriptional control of a constitutive promoter. In another embodiment, the replication deficient vector comprises a second gene under the transcriptional control of a TRE comprising an HRE. In one example, the second gene is an adenovirus gene, for example, an early gene. In another example, the second gene is a suicide gene, including but not limited to TNF-α, Trail, Bax, HSV-tk, cytosine deaminase, p450, diphtheria toxin, a soluable FLT1 gene, and an extracellular FLK-1 gene. In yet another example, the second gene is an immunostimulatory molecule, including but not limited to IL2 and IL12.

Accordingly, it is an object of the presently claimed subject matter to provide a therapeutic method that employs conditional replication of an adenovirus vector in a target tissue expressing hypoxia inducible factor 1 (HIF-1). This and other objects are achieved in whole or in part by the presently claimed subject matter.

An object of the presently claimed subject matter having been stated above, other objects and advantages of the presently claimed subject matter will become apparent to those of ordinary skill in the art after a study of the following description of the presently claimed subject matter and non-limiting Examples.

### BRIEF DESCRIPTION OF THE DRAWINGS

FIG. 1 is a schematic representation of plasmid vector HRP-EGFP. This vector was used to produce stably transduced cell lines that express EGFP under hypoxic conditions. It contains the EGFP gene under the control of a hypoxia responsive promoter.

FIG. 2 is a schematic representation of conditionally replication competent adenovirus vector AdHRP-E1A-dsRed2. This vector has the E1A gene is under the control of the HRP promoter and constitutively expresses a red fluorescent protein reporter, dsRed2.

FIG. 3 is a schematic representation of an exemplary conditionally replication competent adenovirus vector, AdHRP-E4-dsRed2, where only the E4 gene is under the control of the HRP promoter. This vector also constitutively expresses a red fluorescent protein reporter.

FIG. 4 is a schematic representation of an exemplary conditionally replication competent adenovirus vector, AdHRP-E1AE4-dsRed2, where both the E1A and E4 genes are under the control of the HRP promoter. This vector also constitutively expresses a red fluorescent protein reporter.

FIG. 5 is a schematic representation of adenovirus vectors AdCMV-EGFP and AdCMV-dsRed2. Each vector is a replication deficient adenovirus vector that has a fluorescent marker under the transcriptional control of a constitutive CMV promoter. The vectors are replication deficient by virtue of the presence of deletions in the coding sequences for the E3 polypeptide (depicted by box ΔE3), and additionally, the presence of the CMV-marker construct interrupting the E1 polypeptide coding sequence (depicted by ΔE1).

FIGS. 6A and 6B show the results of treating a xenograft tumor model in mice with an adenovirus vector of the presently claimed subject matter.

FIG. 6A depicts adenovirus vector AdHRPE1A-TNFα. This vector has the adenovirus E1A gene operable linked to an HRP. In addition, it has the tumor necrosis factor-alpha (TNF-α) gene operably linked to a constitutive CMV promoter.

FIG. 6B is a graph showing the ability of intratumorally-injected Ad-HRPE1A-TNF-α to inhibit tumor growth in this xenograft model. Tumor-bearing mice were injected with either a replication deficient control vector (AdCMV-dsRed2; see FIG. 5; solid squares) or AdHRPE1A-TNF-α (solid triangles), and tumor volumes were measured at the time points indicated 25 and compared to tumor volumes on day 0 (volume at day 0 set at 1.0).

### DETAILED DESCRIPTION

The presently claimed subject matter generally relates to methods for propagating a conditionally replication competent adenovirus vector in a cell that expresses the transcription factor hypoxia inducible factor 1 (HIF-1). In one embodiment, the methods involve infecting hypoxic cells, for example a hypoxic cell in a tumor, with a conditionally replication competent adenovirus vector such that the adenovirus vector replicates in the hypoxic cell, killing the cell.

I. General Considerations

Hypoxia, a state of lower than normal tissue oxygen tension, has recently been implicated in a host of human diseases, including cancer. It is prominently involved in tumor growth and development. Specifically, hypoxia is found to play a critical role in promoting mutagenesis and selecting for malignant tumor cells. It is also involved in promoting tumor angiogenesis.

Cellular responses to hypoxia are primarily mediated by the transcription factor hypoxia inducible factor 1 (HIF-1). Under conditions of low oxygen, HIF-1 binds to sequences called hypoxia responsive elements (HREs) that are present in the promoters of certain hypoxia responsive genes. The binding of HIF-1 to an HRE-containing promoter results in up-regulated transcription of the associated gene.

The active form of HIF-1 is a heterodimer composed of a regulatory component (HIF-1α) and the constitutively expressed aryl hydrocarbon receptor nuclear translocator (ARNT, also called HIF-1β). The regulation of HIF-1-mediated transcription occurs via post-translational modifications of HIF-1α that depend upon the oxygen status of the cell. Under normoxic conditions, HIF-1α is hydroxylated by the enzyme prolyl hydroxylase using molecular oxygen as the oxygen donor. This hydroxylation allows von Hippel-Lindau protein (pVHL), which is normally present within the cell, to bind to HIF-1α, forming a pVHL/HIF-1α complex. The pVHL/HIF-1α complex is subject to ubiquitylation and degradation in the proteasome. Under hypoxic conditions, on the other hand, prolyl hydroxylase activity is much lower due to the relative absence of the oxygen donor. Under these conditions, HIF-1α is not hydroxylated, VHL/HIF-1α complexes do not form, and the steady state level of HlF-1α within the cell increases. HIF-1α is thus available to form active HIF-1 by complexing with HIF-1β, which results in the transcription of those genes with HRE-containing promoters.

HIF-1 binding results in increased expression of several genes, including transcription factors, growth factors, and cytokines, as well as genes involved in oxygen transport and iron metabolism, glycolysis and glucose uptake, and stress-response. In addition, hypoxia regulates cellular proliferation and migration related to angiogenesis. The vascular endothelial growth factor (VEGF) gene, the product of which is a critical regulatory factor in angiogenesis, contains an HRE in its promoter. HIF-1 upregulates the expression of VEGF and FLT-1, a VEGF receptor. Due to the high growth rate of the cells that make up a solid tumor, new blood vessels are constantly needed to provide rapidly growing tumor cells with adequate nutrients, including oxygen. These newly formed blood vessels frequently are characterized by abnormalities, such that it is very common to find areas of tumors in which individual cells fail to be oxygenated sufficiently. In fact, data suggests that there are localized regions of hypoxia in virtually every tumor larger than 1 mm³ (Dachs & Tozer, 2000).

II. Definitions

While the following terms are believed to be well understood by one of ordinary skill in the art, the following definitions are set forth to facilitate explanation of the presently claimed subject matter.

II.A. Nucleic Acids

The nucleic acid molecules employed in accordance with the presently claimed subject matter include but are not limited to the isolated nucleic acid molecules of any one of SEQ ID NOs:1 and 2; sequences substantially identical to sequences of any one of SEQ ID NOs:1 and 2; conservative variants thereof, subsequences and elongated sequences thereof, complementary DNA molecules, and corresponding RNA molecules. The presently claimed subject matter also encompasses genes, cDNAs, chimeric genes, and vectors comprising disclosed nucleic acid sequences.

The term “nucleic acid molecule” refers to deoxyribonucleotides or ribonucleotides and polymers thereof in either single- or double-stranded form. Unless specifically limited, the term encompasses nucleic acids containing known analogues of natural nucleotides that have similar properties as the reference natural nucleic acid. Unless otherwise indicated, a particular nucleotide sequence also implicitly encompasses conservatively modified variants thereof (e.g., degenerate codon substitutions), complementary sequences, subsequences, elongated sequences, as well as the sequence explicitly indicated. The terms “nucleic acid molecule” or “nucleotide sequence” can also be used in place of “gene”, “cDNA”, or “mRNA”. Nucleic acids can be derived from any source, including any organism.

The term “isolated”, as used in the context of a nucleic acid molecule, indicates that the nucleic acid molecule exists apart from its native environment and is not a product of nature. An isolated DNA molecule can exist in a purified form or can exist in a non-native environment such as a transgenic host cell.

The term “substantially identical”, in the context of two nucleotide sequences, refers to two or more sequences or subsequences that in one example have at least 60%, in another example about 70%, in another example about 80%, in another example about 90-95%, and in yet another example about 99% nudeotide identity, when compared and aligned for maximum correspondence, as measured using one of the following sequence comparison algorithms (described herein below under the heading “Nucleotide and Amino Acid Sequence Comparisons” or by visual inspection. In one example, the substantial identity exists in nucleotide sequences of at least 50 residues, in another example in nucleotide sequence of at least about 100 residues, in another example in nucleotide sequences of at least about 150 residues, and in yet another example in nucleotide sequences comprising complete coding sequences. In one aspect, polymorphic sequences can be substantially identical sequences. The term “polymorphic” refers to the occurrence of two or more genetically determined alternative sequences or alleles in a population. An allelic difference can be as small as one base pair.

Another indication that two nucleotide sequences are substantially identical is that the two molecules specifically or substantially hybridize to each other under stringent conditions. In the context of nucleic acid hybridization, two nucleic acid sequences being compared can be designated a “probe” and a “target”. A “probe” is a reference nucleic acid molecule, and a “target” is a test nucleic acid molecule, often found within a heterogeneous population of nucleic acid molecules. A “target sequence” is synonymous with a “test sequence”.

An exemplary nucleotide sequence employed for hybridization studies or assays includes probe sequences that are complementary to or mimic in one embodiment at least an about 14 to 40 nucleotide sequence of a nucleic acid molecule of the presently claimed subject matter. In one example, probes comprise 14 to 20 nucleotides, or even longer where desired, such as 30, 40, 50, 60, 100, 200, 300, or 500 nucleotides or up to the full length of any of those set forth as SEQ ID NOs:1 and 2. Such fragments can be readily prepared by, for example, directly synthesizing the fragment by chemical synthesis, by application of nucleic acid amplification technology, or by introducing selected sequences into recombinant vectors for recombinant production. The phrase “hybridizing specifically to” refers to the binding, duplexing, or hybridizing of a molecule only to a particular nucleotide sequence under stringent conditions when that sequence is present in a complex nucleic acid mixture (e.g., total cellular DNA or RNA). The phrase “hybridizing substantially to” refers to complementary hybridization between a probe nucleic acid molecule and a target nucleic acid molecule and embraces minor mismatches that can be accommodated by reducing the stringency of the hybridization media to achieve the desired hybridization.

“Stringent hybridization conditions” and “stringent hybridization wash conditions” in the context of nucleic acid hybridization experiments such as Southern and Northern blot analysis are both sequence- and environment-dependent. Longer sequences hybridize specifically at higher temperatures. An extensive guide to the hybridization of nucleic acids is found in Tijssen, 1993. Generally, highly stringent hybridization and wash conditions are selected to be about 5° C. lower than the thermal melting point (Tₘ) for the specific sequence at a defined ionic strength and pH. Typically, under “stringent conditions” a probe will hybridize specifically to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.1×SSC, SM NaCl at 65° C. An example of stringent wash conditions is 15 minutes in 0.2×SSC buffer at 65° C. (see Sambrook and Russell, 2001 for a description of SSC buffer). Often, a high stringency wash is preceded by a low stringency wash to remove background probe signal. An example of medium stringency wash conditions for a duplex of more than about 100 nucleotides, is 15 minutes in 1×SSC at 45° C. An example of low stringency wash for a duplex of more than about 100 nucleotides, is 15 minutes in 4-6×SSC at 40° C. For short probes (e.g., about 10 to 50 nucleotides), stringent conditions typically involve salt concentrations of less than about 1M Na⁺ ion, typically about 0.01 to 1M Na⁺ ion concentration (or other salts) at pH 7.0-8.3, and the temperature is typically at least about 30° C. Stringent conditions can also be achieved with the addition of destabilizing agents such as formamide. In general, a signal to noise ratio of 2-fold (or higher) than that observed for an unrelated probe in the particular hybridization assay indicates detection of a specific hybridization.

The following are examples of hybridization and wash conditions that can be used to clone homologous nucleotide sequences that are substantially identical to reference nucleotide sequences of the presently claimed subject matter a probe nucleotide sequence hybridizes in one example to a target nucleotide sequence in 7% sodium dodecyl sulfate (SDS), 0.5M NaPO₄, 1 mm EDTA at 50° C. followed by washing in 2×SSC, 0.1% SDS at 50° C.; in another example, a probe and target sequence hybridize in 7% sodium dodecyl sulfate (SDS), 0.5M NaPO₄, 1 mm EDTA at 50° C. followed by washing in 1×SSC, 0.1% SDS at 50° C.; in another example, a probe and target sequence hybridize in 7% sodium dodecyl sulfate (SDS), 0.5M NaPO₄, 1 mm EDTA at 50° C. followed by washing in 0.5×SSC, 0.1% SDS at 50° C.; in another example, a probe and target sequence hybridize in 7% sodium dodecyl sulfate (SDS), 0.5M NaPO₄, 1 mm EDTA at 50° C. followed by washing in 0.1×SSC, 0.1% SDS at 50° C.; in yet another example, a probe and target sequence hybridize in 7% sodium dodecyl sulfate (SDS), 0.5M NaPO₄, 1 mm EDTA at 50° C. followed by washing in 0.1×SSC, 0.1% SDS at 65° C.

A further indication that two nucleic acid sequences are substantially identical is that proteins encoded by the nucleic acids are substantially identical, share an overall three-dimensional structure, are biologically functional equivalents, or are immunologically cross-reactive. These terms are defined further under the heading “Polypeptides” herein below. Nucleic acid molecules that do not hybridize to each other under stringent conditions are still substantially identical if the corresponding proteins are substantially identical. This can occur, for example, when two nucleotide sequences are significantly degenerate as permitted by the genetic code.

The term “conservatively substituted variants” refers to nucleic acid sequences having degenerate codon substitutions wherein the third position of one or more selected (or all) codons is substituted with mixed-base and/or deoxyinosine residues (Ohtsuka et al., 1985; Batzer et al., 1991; Rossolini et al., 1994)

The term “subsequence” refers to a sequence of nucleic acids that comprises a part of a longer nucleic acid sequence. An exemplary subsequence is a probe, described herein above, or a primer. The term “primer” as used herein refers to a contiguous sequence comprising in one example about 8 or more deoxyribonucleotides or ribonucleotides, in another example 10-20 nucleotides, and in yet another example 20-30 nucleotides of a selected nucleic acid molecule. The primers of the presently claimed subject matter encompass oligonucleotides of sufficient length and appropriate sequence so as to provide initiation of polymerization on a nucleic acid molecule of the presently claimed subject matter.

The term “elongated sequence” refers to an addition of nucleotides (or other analogous molecules) incorporated into the nucleic acid. For example, a polymerase (e.g., a DNA polymerase) can add sequences at the 3′ terminus of the nucleic acid molecule. In addition, the nucleotide sequence can be combined with other DNA sequences, such as promoters, promoter regions, enhancers, polyadenylation signals, intronic sequences, additional restriction enzyme sites, multiple cloning sites, and other coding segments.

The term “complementary sequences”, as used herein, indicates two nucleotide sequences that comprise antiparallel nucleotide sequences capable of pairing with one another upon formation of hydrogen bonds between base pairs. As used herein, the term “complementary sequences” means nucleotide sequences which are substantially complementary, as can be assessed by the same nucleotide comparison set forth above, or is defined as being capable of hybridizing to the nucleic acid segment in question under relatively stringent conditions such as those described herein. A particular example of a complementary nucleic acid segment is an antisense oligonucleotide.

The term “gene” refers broadly to any segment of DNA associated with a biological function. A gene encompasses sequences including but not limited to a coding sequence, a promoter region, a transcriptional regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

The term “gene expression” generally refers to the cellular processes by which a biologically active polypeptide is produced from a DNA sequence.

The presently claimed subject matter can also employ chimeric genes. The term “chimeric gene”, as used herein, refers to a promoter region operatively linked to a nucleotide sequence encoding a therapeutic polypeptide; a nucleotide sequence producing an antisense RNA molecule; a RNA molecule having tertiary structure, such as a hairpin structure; or a double-stranded RNA molecule.

The terms “operatively linked” and “operably linked”, as used herein, refer to a promoter region that is connected to a nucleotide sequence in such a way that the transcription of that nucleotide sequence is controlled and regulated by that promoter region. Similarly, a nucleotide sequence is said to be under the “transcriptional control” of a promoter to which it is operably linked. Techniques for operatively linking a promoter region to a nucleotide sequence are known in the art.

The terms “heterologous gene”, “heterologous DNA sequence”, “heterologous nucleotide sequence”, “exogenous nucleic acid molecule”, or “exogenous DNA segment”, as used herein, each refer to a sequence that originates from a source foreign to an intended host cell or, if from the same source, is modified from its original form. Thus, a heterologous gene in a host cell includes a gene that is endogenous to the particular host cell but has been modified, for example by mutagenesis or by isolation from native transcriptional regulatory sequences. The terms also include non-naturally occurring multiple copies of a naturally occurring nucleotide sequence. Thus, the terms refer to a DNA segment that is foreign or heterologous to the cell, or homologous to the cell but in a position within the host cell nucleic acid wherein the element is not ordinarily found.

The term “construct” as used herein means a DNA sequence capable of directing expression of a particular nucleotide sequence in an appropriate host cell, comprising a promoter operatively linked to the nucleotide sequence of interest which is operatively linked to termination signals. It also typically comprises sequences required for proper translation of the nucleotide sequence. The construct comprising the nucleotide sequence of interest can be chimeric. The construct can also be one that is naturally occurring but has been obtained in a recombinant form useful for heterologous expression.

The term “promoter” or “promoter region” each refers to a nucleotide sequence within a gene that is positioned 5′ to a coding sequence of a same gene and functions to direct transcription of the coding sequence. The promoter region comprises a transcriptional start site, and can additionally include one or more transcriptional regulatory elements. In one embodiment, a method of the presently claimed subject matter employs a hypoxia inducible promoter.

A “minimal promoter” is a nucleotide sequence that has the minimal elements required to enable basal level transcription to occur. As such, minimal promoters are not complete promoters but rather are subsequences of promoters that are capable of directing a basal level of transcription of a reporter construct in an experimental system. Minimal promoters include but are not limited to the CMV minimal promoter, the HSV-tk minimal promoter, the simian virus 40 (SV40) minimal promoter, the human b-actin minimal promoter, the human EF2 minimal promoter, the adenovirus E1B minimal promoter, and the heat shock protein (hsp) 70 minimal promoter. Minimal promoters are often augmented with one or more transcriptional regulatory elements to influence the transcription of an operably linked gene. For example, cell-type-specific or tissue-specific transcriptional regulatory elements can be added to minimal promoters to create recombinant promoters that direct transcription of an operably linked nucleotide sequence in a cell-type-specific or tissue-specific manner. In one embodiment of the presently claimed subject matter, a hypoxia inducible promoter comprises the CMV minimal promoter linked to five tandem copies of the HRE from the human VEGF promoter.

Different promoters have different combinations of transcriptional regulatory elements. Whether or not a gene is expressed in a cell is dependent on a combination of the particular transcriptional regulatory elements that make up the gene's promoter and the different transcription factors that are present within the nucleus of the cell. As such, promoters are often classified as “constitutive”, “tissue-specific”, “cell-type-specific”, or “inducible”, depending on their functional activities in vivo or in vitro. For example, a constitutive promoter is one that is capable of directing transcription of a gene in a variety of cell types. Exemplary constitutive promoters include the promoters for the following genes which encode certain constitutive or “housekeeping” functions: hypoxanthine phosphoribosyl transferase (HPRT), dihydrofolate reductase (DHFR; (Scharfmann et al, 1991), adenosine deaminase, phosphoglycerate kinase (PGK), pyruvate kinase, phosphoglycerate mutase, the β-actin promoter (see e.g., Williams et at, 1993), and other constitutive promoters known to those of skill in the art. “Tissue-specific” or “cell-type-specific” promoters, on the other hand, direct transcription in some tissues and cell types but are inactive in others. Exemplary tissue-specific promoters include the PSA promoter (Yu et al., 1999; Lee et al., 2000), the probasin promoter (Greenberg et al, 1994; Yu et al., 1999), and the MUC1 promoter (Kurihara et al., 2000) as discussed above, as well as other tissue-specific and cell-type specific promoters known to those of skill in the art.

An “inducible” promoter is one for which the transcription level of an operably linked gene varies based on the presence of a certain stimulus. Genes that are under the control of inducible promoters are expressed only, or to a greater degree, in the presence of an inducing agent, (e.g., transcription under control of the metallothionein promoter is greatly increased in presence of certain metal ions). Inducible promoters include transcriptional regulatory elements (TREs), which stimulate transcription when their inducing factors are bound. For example, there are TREs for serum factors, steroid hormones, retinoic acid and cyclic AMP. Promoters containing a particular TRE can be chosen in order to obtain an inducible response, and in some cases, the TRE itself can be attached to a different promoter, thereby conferring inducibility to the recombinant gene. In one embodiment of the presently claimed subject matter, an adenovirus vector comprises a hypoxia inducible promoter that confers HIF-1-mediated inducibility to an adenovirus gene.

As used herein, the term “hypoxia inducible promoter” refers to a promoter that contains hypoxia responsive elements such that the active form of HIF-1, if present, will bind and cause the transcription of an operably linked nucleotide sequence to be enhanced above basal levels. As such, a hypoxia inducible promoter is one from which under normoxic conditions an operably linked nucleotide sequence is transcribed at basal levels or below due to the absence of active HIF-1.

In addition, as used herein with regard to the presently claimed subject matter, the presence of active HIF-1 in a cell includes not only conditions wherein the cell experiences hypoxia, but also includes any other condition where the active form of HIF-1 accumulates and is available to bind an HRE. Such other conditions include conditions wherein the interaction between HIF-1α and pVHL, and hence the ubiquitylation and degradation of HIF-1α, does not occur. For example, active HIF-1 can be formed as a result of a modification in the activity of a prolyl hydroxylase polypeptide (e.g. a mutation) such that the hydroxylation of HIF-1α does not occur. Alternatively, in cells that lack pVHL, active HIF-1 accumulates (see e.g., Clifford & Maher, 2001). “Normoxic conditions” or “normoxia” refer to a state of normal oxygen saturation in which the HIF-1α polypeptide is hydroxylated by prolyl hydroxylase as described above, and thus a cell does not accumulate the active form of HIF-1.

When used in the context of a promoter, the term “linked” as used herein refers to a physical proximity of promoter elements such that they function together to direct transcription of an operably linked nucleotide sequence. In one embodiment of the presently claimed subject matter, a minimal promoter is linked to an HRE, resulting in hypoxia inducible transcription of an adenovirus gene in a cell containing active HIF-1 transcription factor.

The term “transcriptional regulatory sequence” or “transcriptional regulatory element”, as used herein, each refers to a nucleotide sequence within the promoter region that enables responsiveness to a regulatory transcription factor. Responsiveness can encompass a decrease or an increase in transcriptional output and is mediated by binding of the transcription factor to the DNA molecule comprising the transcriptional regulatory element. In one example, a transcriptional regulatory element is an HRE.

The term “transcription factor” generally refers to a protein that modulates gene expression by interaction with the transcriptional regulatory element and cellular components for transcription, including RNA Polymerase, Transcription Associated Factors (TAFs), chromatin-remodeling proteins, and any other relevant protein that impacts gene transcription.

The terms “reporter gene” or “marker gene” or “selectable marker” each refer to a heterologous gene encoding a product that is readily observed and/or quantitated. A reporter gene is heterologous in that it originates from a source foreign to an intended host cell or, if from the same source, is modified from its original form. Non-limiting examples of detectable reporter genes that can be operatively linked to a transcriptional regulatory region can be found in Alam & Cook (1990) Anal Biochem 188:245-254 and PCT International Publication No. WO 97/47763. Exemplary reporter genes for transcriptional analyses include the lacZ gene (see e.g., Rose & Botstein (1983) Meth Enzymol 101:167-180), Green Fluorescent Protein (GFP; Cubift et al., 1995), luciferase, and chloramphenicol acetyl transferase (CAT). Reporter genes for methods to produce transgenic animals include but are not limited to antibiotic resistance genes, for example the antibiotic resistance gene confers neomycin resistance. Any suitable reporter and detection method can be used, and it will be appreciated by one of skill in the art that no particular choice is essential to or a limitation of the presently claimed subject matter.

An amount of reporter gene can be assayed by any method for qualitatively or quantitatively determining presence or activity of the reporter gene product. The amount of reporter gene expression directed by each test promoter region fragment is compared to an amount of reporter gene expression to a control construct comprising the reporter gene in the absence of a promoter region fragment. A promoter region fragment is identified as having promoter activity when there is significant increase in an amount of reporter gene expression in a test construct as compared to a control construct. The term “significant increase”, as used herein, refers to an quantified change in a measurable quality that is larger than the margin of error inherent in the measurement technique, in one example an increase by about 2-fold or greater relative to a control measurement, in another example an increase by about 5-fold or greater, and in yet another example an increase by about 10-fold or greater.

The presently claimed subject matter further includes adenovirus vectors comprising the disclosed nucleotide sequences. The term “vector”, as used herein refers to a DNA molecule having sequences that enable the transfer of those sequences to a compatible host cell. A vector also includes nucleotide sequences to permit ligation of nucleotide sequences within the vector, wherein such nucleotide sequences are also replicated in a compatible host cell. A vector can also mediate recombinant production of a therapeutic polypeptide, as described further herein below.

Nucleic acids of the presently claimed subject matter can be cloned, synthesized, recombinantly altered, mutagenized, or combinations thereof. Standard recombinant DNA and molecular cloning techniques used to isolate nucleic acids are known in the art. Exemplary, non-limiting methods are described by Silhavy et al., 1984; Ausubel et al., 1992; Glover & Hames, 1995; and Sambrook & Russell, 200). Site-specific mutagenesis to create base pair changes, deletions, or small insertions is also known in the art as exemplified by publications (see e.g., Adelman et al., 1983; Sambrook & Russell, 2001).

II.B. Polypeptides

The polypeptides employed in accordance with the presently claimed subject matter include but are not limited to a therapeutic polypeptide as defined herein below; a polypeptide substantially identical to a therapeutic polypeptide as defined herein below, a polypeptide fragment of a therapeutic polypeptide as defined herein below (in one embodiment biologically functional fragments), fusion proteins comprising a therapeutic polypeptide as defined herein below, biologically functional analogs thereof, and polypeptides that cross-react with an antibody that specifically recognizes a therapeutic polypeptide as defined herein below. The polypeptides employed in accordance with the presently claimed subject matter include but are not limited to isolated polypeptides, polypeptide fragments, fusion proteins comprising the disclosed amino acid sequences, biologically functional analogs, and polypeptides that cross-react with an antibody that specifically recognizes a disclosed polypeptide.

The term “isolated”, as used in the context of a polypeptide, indicates that the polypeptide exists apart from its native environment and is not a product of nature. An isolated polypeptide can exist in a purified form or can exist in a non-native environment such as, for example, in a transgenic host cell.

The term “substantially identical” in the context of two or more polypeptide sequences is measured as polypeptide sequences having in one example about 35%, or 45%, in another example from 45-55%, and in another example 55-65% of identical or functionally equivalent amino acids. In another example, two or more “substantially identical” polypeptide sequences will have about 70%, or in another example about 80%, in another example about 90%, in another example about 95%, and in yet another example about 99% identical or functionally equivalent amino acids. Percent “identity” and methods for determining identity are defined herein below under the heading “Nucleotide and Amino Acid Sequence Comparisons”.

Substantially identical polypeptides also encompass two or more polypeptides sharing a conserved three-dimensional structure. Computational methods can be used to compare structural representations, and structural models can be generated and easily tuned to identify similarities around important active sites or ligand binding sites (see Barton, 1998; Saqi et al., 1999; Henikoff et al., 2000; Huang et al., 2000).

The term “functionally equivalent” in the context of amino acid sequences is known in the art and is based on the relative similarity of the amino acid side-chain substituents (see Henikoff & Henikoff, 2000). Relevant factors for consideration include side-chain hydrophobicity, hydrophilicity, charge, and size. For example, arginine, lysine, and histidine are all positively charged residues; that alanine, glycine, and serine are all of similar size; and that phenylalanine, tryptophan, and tyrosine all have a generally similar shape. By this analysis, described further herein below, arginine, lysine, and histidine; alanine, glycine, and serine; and phenylalanine, tryptophan, and tyrosine; are defined herein as biologically functional equivalents.

In making biologically functional equivalent amino acid substitutions, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte & Doolittle, 1982). It is known that certain amino acids can be substituted for other amino acids having a similar hydropathic index or score and still retain a similar biological activity. In making changes based upon the hydropathic index, the substitution of amino acids involves in one example those with hydropathic indices within ±2 of the original value, in another example those within ±1 of the original value, and in yet another example those within ±0.5 of the original value.

It is also understood in the art that the substitution of like amino acids can be made effectively on the basis of hydrophilicity. U.S. Pat. No. 4,554,101 states that the greatest local average hydrophilicity of a protein, as governed by the hydrophilicity of its adjacent amino acids, correlates with its immunogenicity and antigenicity, e.g., with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids is in one example those with hydrophilicity values within ±2 of the original value, in another example those within ±1 of the original value, and in yet another example those within ±0.5 of the original value.

The methods of the presently claimed subject matter can also employ polypeptide fragments or functional portions of a polypeptide, such as an interleukin polypeptide. Such functional portion need not comprise all or substantially all of the amino acid sequence of a native gene product. The term “functional” includes any biological activity or feature of the polypeptide. In the case of an interleukin polypeptide, the biological activity is for example an immunostimulatory or anti-angiogenic activity in vivo as disclosed herein.

The presently claimed subject matter also includes longer sequences of a therapeutic polypeptide. For example, one or more amino acids can be added to the N-terminus or C-terminus of the polypeptide. Fusion proteins comprising therapeutic polypeptide sequences (for example, interleukin polypeptide sequences) are also provided within the scope of the presently claimed subject matter. Methods of preparing such proteins are known in the art. In one example, the fusion protein includes any biological activity of a therapeutic polypeptide. In the case of an interleukin polypeptide, the biological activity is in one embodiment any biological activity of a native interleukin, for example, an immunostimulatory or anti-angiogenic activity in vivo as disclosed herein. Optionally, a fusion protein can have additional biological activities provided by the fused heterologous sequence.

The presently claimed subject matter also encompasses functional analogs of a therapeutic polypeptide. Functional analogs share at least one biological function with a therapeutic polypeptide (for example, an interleukin polypeptide). In the context of amino acid sequence, biologically functional analogs, as used herein, are peptides in which certain, but not most or all, of the amino acids can be substituted. Functional analogs can be created at the level of the corresponding nucleic acid molecule, altering such sequence to encode desired amino acid changes. In one embodiment, changes can be introduced to improve a biological function of the polypeptide, e.g., to improve the therapeutic effectiveness of the polypeptide (for example, an interleukin polypeptide).

The presently claimed subject matter also encompasses recombinant production of the disclosed polypeptides. Briefly, a nucleic acid sequence encoding a therapeutic polypeptide, is cloned into a construct, the construct is introduced into a host organism, where it is recombinantly produced.

The term “host organism” refers to any organism into which a disclosed adenovirus vector has been introduced. In one embodiment, the host organism is a warm-blooded vertebrate, in another embodiment, a mammal.

II.C. Nucleotide and Amino Acid Sequence Comparisons

The terms “identical” or percent “identity” in the context of two or more nucleotide or polypeptide sequences, refer to two or more sequences or subsequences that are the same or have a specified percentage of amino acid residues or nucleotides that are the same, when compared and aligned for maximum correspondence, as measured using one of the sequence comparison algorithms disclosed herein or by visual inspection.

The term “substantially identical” in regards to a nucleotide or polypeptide sequence means that a particular sequence varies from the sequence of a naturally occurring sequence by one or more deletions, substitutions, or additions, the net effect of which is to retain at least some of biological activity of the natural gene, gene product, or sequence. Such sequences include “mutant” sequences, or sequences wherein the biological activity is altered to some degree but retains at least some of the original biological activity. The term “naturally occurring”, as used herein, is used to describe a composition that can be found in nature as distinct from being artificially produced by man. For example, a protein or nucleotide sequence present in an organism, which can be isolated from a source in nature and which has not been intentionally modified by man in the laboratory, is naturally occurring.

For sequence comparison, typically one sequence acts as a reference sequence to which test sequences are compared. When using a sequence comparison algorithm, test and reference sequences are entered into a computer program, subsequence coordinates are designated if necessary, and sequence algorithm program parameters are selected. The sequence comparison algorithm then calculates the percent sequence identity for the designated test sequence(s) relative to the reference sequence, based on the selected program parameters.

Optimal alignment of sequences for comparison can be conducted, e.g., by the local homology algorithm of Smith & Waterman (1981), by the homology alignment algorithm of Needleman & Wunsch (1970), by the search for similarity method of Pearson & Lipman (1988), by computerized implementations of these algorithms (GAP, BESTFIT, FASTA, and TFASTA in the GCG® WISCONSIN PACKAGE®, available from Accelrys, Inc., San Diego, Calif., United States of America), or by visual inspection (see generally, Ausubel et al., 1992).

An exemplary algorithm for determining percent sequence identity and sequence similarity is the BLAST algorithm, which is described in Altschul et al., 1990. Software for performing BLAST analyses is publicly available through the National Center for Biotechnology Information (http://www.ncbi.nim.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength W=11, an expectation E=10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, 1992).

In addition to calculating percent sequence identity, the BLAST algorithm also performs a statistical analysis of the similarity between two sequences (see e.g., Karlin & Altschul, 1993). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1 in one example, less than about 0.01 in another example, and less than about 0.001 in yet another example.

III. Adenovirus Vectors

In one embodiment, an adenovirus vector of the presently claimed subject matter is conditionally replication competent. That is, they contain one or more functional genes required for their replication placed under the transcriptional control of an inducible promoter. This retards uncontrolled replication in vivo and reduces undesirable side effects of viral infection. Replication competent self-limiting or self-destructing viral vectors can also be used, as can replication deficient viral vectors.

Incorporation of a nucleic acid construct into a viral genome can be optionally performed by ligating the construct into an appropriate restriction site in the genome of the virus. Viral genomes can then be packaged into viral coats or capsids by any suitable procedure. In particular, any suitable packaging cell line can be used to generate viral vectors of the presently claimed subject matter. These packaging lines complement the conditionally replication deficient viral genomes of the presently claimed subject matter, as they include, typically incorporated into their genomes, the genes which have been put under an inducible promoter deleted in the conditionally replication competent vectors. Thus, the use of packaging lines allows viral vectors of the presently claimed subject matter to be generated in culture.

The adenovirus vectors of the presently claimed subject matter are designed to replicate preferentially in a cell expressing high levels of HIF-1, including, but not limited to, a cell present in a hypoxic region of a tumor. This can be accomplished by putting an adenovirus gene essential for replication under the transcriptional control of a hypoxia responsive promoter (HRP). The ability of this promoter to preferentially direct transcription in hypoxic cells was assessed by producing a plasmid that contained the promoter operatively linked to an enhanced green fluorescent protein (EGFP) coding sequence as described in Example 1. The HRP-EGFP construct was used to establish stable sublines from two tumor cell lines: HCT116, a human colon carcinoma cell line; and 4T1, a murine mammary adenocarcinoma. Cells grown in normoxic conditions failed to express EGFP. Cells from stably transduced sublines exposed to hypoxic conditions (with oxygen tension at 0.5 to 1.5%), showed robust expression of EGFP 24 hours after incubation.

In one embodiment, conditional replication competence using intratumorally-injected constructs provides vector replication in hypoxic regions of a tumor. A feature of the presently claimed subject matter pertains to a method for effectively focusing vector distribution and replication to the hypoxic cells in the vicinity of the site of provision. In vivo intratumoral replication of an adenoviral reporter gene construct was assessed in a subcutaneous tumor model as described in Example 2.

The ability of the HRP to limit transcription to hypoxic cells was tested in vivo by establishing subcutaneous tumors in mice with stable sublines as described in Example 2. Subcutaneous tumors were allowed to grow to 1.0-1.5 cm in diameter. Tumors were then removed from sacrificed mice and EGFP expression was detected. The EGFP reporter gene was expressed exclusively in hypoxic regions of the tumors.

In an effort to maximize intratumoral replication of a hypoxia inducible vector, and concomitantly minimize potentially immunogenic systemic replication of the same vector, constructs were developed that employ a hypoxia responsive promoter. As disclosed herein, high intratumoral replication of the vector can be achieved while replication in surrounding cells is substantially eliminated. Thus, in one embodiment of the presently claimed subject matter, HIF-1 inducible replication of an adenovirus vector in a tumor can result in suppression of tumor growth.

Any hypoxia inducible promoter can be used in accordance with the methods of the presently claimed subject matter, including but not limited to a recombinant promoter comprising a minimal promoter linked to an HIF-1 binding sequence. In one example, an HIF-1 binding sequence is an HRE. HREs have been found in the promoters of several hypoxia inducible genes, including phosphoglycerate kinase-1 (Firth et al., 1994; Semenza et al., 1994), erythropoietin (Pugh et al., 1991; Semenza et al., 1991), and VEGF (Liu et al., 1995; Forsythe et al., 1996).

For genes that are upregulated in response to hypoxia, wherein the precise sequence that confers hypoxia inducibility has not been determined, the responsive sequence can be defined by methods known to one of ordinary skill in the art. Within a candidate promoter region, the presence of regulatory proteins bound to a nucleic acid sequence can be detected using a variety of methods well known to those skilled in the art (Ausubel et al., 1992). Briefly, in vivo footprinting assays demonstrate protection of DNA sequences from chemical and enzymatic modification within living or permeabilized cells. Similarly, in vitro footprinting assays show protection of DNA sequences from chemical or enzymatic modification using protein extracts. Nitrocellulose filter-binding assays and gel electrophoresis mobility shift assays (EMSAs) track the presence of radiolabeled regulatory DNA elements based on provision of candidate transcription factors. Computer analysis programs, for example TFSEARCH version 1.3 (Yutaka Akiyama: “TFSEARCH: Searching Transcription Factor Binding Sites”, http://www.rwcp.or.jp/papia/), can also be used to locate consensus sequences of known transcriptional regulatory elements within a genomic region.

A hypoxia inducible promoter of the presently claimed subject matter can be concatamerized or combined with additional elements to amplify transcriptional activity. In one embodiment of the presently claimed subject matter, the hypoxia inducible promoter comprises five tandem copies of the HRE from the human VEGF gene linked to the CMV minimal promoter.

Alternatively or in addition, the hypoxia inducible promoter can be combined with an element that acts as an enhancer of mRNA translation. In one embodiment, an enhancer of mRNA translation is an HRE.

A hypoxia inducible promoter of the presently claimed subject matter can further be responsive to non-hypoxia stimuli that can be used in combined therapy treatments as disclosed herein. For example, the mortalin promoter is induced by low doses of ionizing radiation (Sadekova et al., 1997), the hsp27 promoter is activated by 17β-estradiol and estrogen receptor agonists (Porter et al., 2001), the HLA-G promoter is induced by arsenite, and hsp promoters can be activated by photodynamic therapy (Luna et al., 2000). Thus, a hypoxia inducible promoter used in accordance with the presently claimed subject matter can comprise additional inducible features or additional DNA elements that support combined therapy treatments. Virus administration can be provided before, during, or after radiotherapy; before, during, or after chemotherapy; and/or before, during, or after photodynamic therapy.

A hypoxia inducible promoter can be derived from any biological source, including from a source that is heterologous to the intended subject to be treated. As one example, the human VEGF promoter can direct efficient hypoxia inducible expression in bovine pulmonary artery endothelial (BPAE) cells (Liu et al., 1995).

IV. Transgenes

The methods of the presently claimed subject matter employ adenovirus vectors to replicate in cells, thereby causing lysis of the cells. In order to more efficiently kill a cell that contains an adenovirus vector, the presently claimed subject matter also provides adenovirus vectors comprising a transgene. In accordance with the presently claimed subject matter, a transgene can comprise a therapeutic gene, including, but not limited to a tumor suppressor gene, an apoptosis-inducing gene, an anti-angiogenic gene, a suicide prodrug converting enzyme gene, a bacterial toxin gene, an antisense gene, a tumor suppressor gene, an immunostimulatory gene, or combinations thereof.

As used herein, the term “transgene” refers to any nucleotide sequence to be introduced into a cell, thereby allowing the nucleotide sequence to be expressed in the cell. A transgene can include a gene that is partly or entirely heterologous (ie. foreign) to the organism from which the cell was derived, or can be a nucleotide sequence identical or homologous to a gene already contained within the cell. In one embodiment of the presently claimed subject matter, a transgene comprises a therapeutic gene.

In one embodiment of the presently claimed subject matter, a transgene is encoded by a conditionally replication competent adenovirus vector. However, the number of exogenous nucleotides that can be efficiently packaged into an adenovirus virion is about 2000 base pairs. Thus, a conditionally replication competent adenovirus vector of the presently claimed subject matter can optionally comprise a transgene of no more than about 1.4-1.6 kilobases (kb), in addition to promoter and polyadenylation sequences that are essential for each transgene. Transgenes larger than this are typically provided by other mechanisms. As disclosed herein below, in one embodiment of the presently claimed subject matter, a method is provided wherein a transgene is delivered by a replication deficient adenovirus vector, which itself becomes replicate-competent in cells where the replication competent virus is present. This occurs because the essential early gene products deleted from the former are provided for by the latter.

The methods of the presently claimed subject matter can be used to cause cell death by adenovirus vector replication, which results in cell lysis. An adenovirus vector of the presently claimed subject matter can additionally include a transgene comprising a nucleic acid molecule that encodes a polypeptide having a therapeutic biological activity (also referred to herein as a “therapeutic polypeptide”). Exemplary therapeutic polypeptides include but are not limited to immunostimulatory molecules, tumor suppressor gene products/antigens, suicide gene products, and anti-angiogenic factors (see Mackensen et al., 1997; Walther & Stein, 1999; Kirk & Mule, 2000 and references cited therein).

Angiogenesis and suppressed immune response play a central role in the pathogenesis of malignant disease and tumor growth, invasion, and metastasis. Thus, in one example, the therapeutic polypeptide has an ability to induce an immune response and/or an anti-angiogenic response in vivo. In one embodiment, an adenovirus vector of the presently claimed subject matter encodes a therapeutic gene that displays both immunostimulatory and anti-angiogenic activities, for example, IL12 (see Dias et al, 1998, and references cited herein below), interferon-α (see O'Byrne et al., 2000, and references cited therein), or a chemokine (see Nomura & Hasegawa, 2000, and references cited therein). In another embodiment, an adenovirus vector of the presently claimed subject matter encodes a gene product with immunostimulatory activity and a gene product having anti-angiogenic activity (see e.g., Narvaiza et al., 2000).

IL12, optionally in combination with the co-stimulatory agent B7.1, is a representative therapeutic polypeptide because local application of virus encoding IL12 or B7.1, as well as the combination of IL12 and B7.1, appear to improve immune responses against tumors (Putzer et al., 1997).

In one embodiment, the presently claimed subject matter comprises an adenovirus vector encoding an IL12 polypeptide capable of eliciting an immune response and/or an anti-angiogenic response. Interleukin-12 (IL12) is a disulfide-linked heterodimer composed of 2 subunits: p35 and p40. IL12 stimulates T and NK cells to secrete interferon-gamma (IFN-γ) and augments T and NK cell proliferation and cytolytic activity (Kobayashi et al., 1989; Wolf et al., 1991; D'Andre et al., 1992; Gately et al., 1994; Robertson et al., 1992). Through these functions, IL12 promotes early inflammatory responses and the development of CD4+ T helper (Th1) cells that favor cell-mediated immunity (Manetti et al., 1993; Hsieh et al., 1993). IL12 further inhibits angiogenesis, possibly through a NK cell-mediated mechanism (Voest et al., 1995; Majewski et al., 1996; Yao et al., 1999). In one example, the IL12 polypeptide encoded by a gene therapy construct of the presently claimed subject matter displays one or more biological properties of a naturally occurring IL12 polypeptide.

In another embodiment, the presently claimed subject matter comprises an adenovirus vector encoding an IL2 polypeptide. IL2 is an immunostimulatory molecule that shows therapeutic activity in a variety of cancers, including renal cancer, breast cancer, bladder cancer, and malignant melanoma. The anti-tumor activity of IL2 is related to its capacity to expand and activate NK cells and T cells that express IL2 receptors (see e.g., Margolin, 2000; Gore, 1996; Deshmukh et al, 2001; Larchian et al., 2000; Horiguchi et al., 2000; and references cited therein. IL2 has also been used successfully when co-administered with anti-tumor vaccines (see Overwijk et al., 2000, and references cited therein).

In one example, the IL2 polypeptide encoded by an adenovirus vector of the presently claimed subject matter displays one or more biological properties of a naturally occurring IL2 polypeptide. IL2-induced proliferation can be measured, for example, by 3H-thymidine incorporation in CTLL-2 cells, as described in European Patent No. 0 439 095. The biological properties of an IL2 polypeptide can further be assessed using methods described in the foregoing publications.

As used herein, the term “suicide gene” refers to a gene that encodes a polypeptide that causes a cell that produces that polypeptide to die. A suicide gene can encode a gene that causes cell death directly, for example by inducing apoptosis. Such a gene is referred to as an “apoptosis-inducing gene”, and includes, but is not limited to TNF-α (Idriss & Naismith, 2000), Trail (Srivastava, 2001), Bax, and Bcl-2 (Shen & White, 2001). Other genes that encode proteins that kill cells directly include bacterial toxin genes, which are normally found in the genome of certain bacteria and encode polypeptides (i.e. bacterial toxins) that are toxic to eukaryotic cells. Bacterial toxins include but are not limited to diphtheria toxin (Frankel et al., 2001).

Alternatively, a suicide gene can encode a polypeptide that converts a prodrug to a toxic compound. Such suicide prodrug converting enzymes include, but are not limited to the HSV-tk polypeptide, which converts ganciclovir to a toxic nucleotide analog (Freeman et al., 1996); cytosine deaminase, which converts the non-toxic nucleotide analog 5-fluorocytosine into a toxic analog, 5-fluorouracil (Yazawa et al., 2002); and cytochrome p450, which converts certain aliphatic amine N-oxides into toxic metabolites (Patterson, 2002).

Additionally, a suicide gene can encode a polypeptide that interferes with a signal transduction cascade involved with cellular survival or proliferation. Such cascades include, but are not limited to, the cascades mediated by the Flt1 and Flk1 receptor tyrosine kinases (reviewed in Klohs, et al., 1997). Polypeptides that can interfere with Flt1 and/or Flk1 signal transduction include, but are not limited to, a soluble Flt1 receptor (s-Flt1; Shibuya, 2001) and an extracellular domain of the Flk-1 receptor (ex-Flk1; Lin et al., 1998).

V. Therapy Methods

A therapeutic method according to the presently claimed subject matter comprises contacting a hypoxic cell in a tumor with an adenovirus vector, whereby the vector enters the cell and inhibits tumor growth. For example, the disclosed adenovirus vectors can be useful in the treatment of both primary and metastatic solid tumors and carcinomas of the breast; colon; rectum; lung; oropharynx; hypopharynx; esophagus; stomach; pancreas; liver; gallbladder; bile ducts; small intestine; urinary tract including kidney, bladder and urothelium; female genital tract including cervix, uterus, ovaries, choriocarcinoma and gestational trophoblastic disease; male genital tract including prostate, seminal vesicles, testes and germ cell tumors; endocrine glands including thyroid, adrenal, and pituitary; skin including hemangiomas, melanomas, sarcomas arising from bone or soft tissues and Kaposi's sarcoma; tumors of the brain, nerves, eyes, and meninges including astrocytomas, gliomas, glioblastomas, retinoblastomas, neuromas, neuroblastomas, Schwannomas and meningiomas; solid tumors arising from hematopoietic malignancies such as leukemias and including chloromas, plasmacytomas, plaques and tumors of mycosis fungoides and cutaneous T-cell lymphoma/leukemia; lymphomas including both Hodgkin's and non-Hodgkin's lymphomas.

The compositions of the presently claimed subject matter can also be useful for the prevention of metastases from the tumors described above either when used alone or in combination with radiotherapeutic, photodynamic, and/or chemotherapeutic treatments conventionally administered to patients for treating disorders, including angiogenic disorders. For example, a tumor can be treated conventionally with surgery, photodynamic therapy, radiation and/or chemotherapy followed by administration of the compositions of the presently claimed subject matter to extend the dormancy of micrometastases and to stabilize and inhibit the growth of any residual primary tumor. Indeed, virus administration can be provided before, during, or after radiotherapy; before, during, or after chemotherapy; and/or before, during, or after photodynamic therapy.

The compositions and methods of the presently claimed subject matter are not limited to use in cells that have elevated HIF-1 expression due to hypoxia. They can also be used in any cell in which an HRE can function to regulate transcription of an operably linked nucleotide sequence. For example, loss of pVHL function has been reported in a familial angiomatous syndrome, and also in the majority of sporadic central nervous system hemangioblastomas and clear cell renal carcinomas (reviewed in Ivan & Kaelin, 2001). Furthermore, pVHL mutations that have been associated with renal cell carcinoma and/or hemangioblastomas have all been shown to interfere with pVHL's ability to regulate HIF-1α activity (Maxwell et al., 2001). Thus, the compositions and methods of the presently claimed subject matter are applicable to cells that have lost pVHL function.

In addition, a recent report suggested that HIF-1 accumulates in some tumor cells even under normoxic conditions. It has long been known that some cancer cells display high rates of glycolysis under aerobic conditions, a phenomenon known as the Warburg effect. Evidence suggests that the Warburg effect is characterized by the accumulation of HIF-1 in transformed cells in normoxic areas of tumors, leading to glycolysis under aerobic conditions. Further, the induction of HIF-1 in these cells appears to be mediated by the pp60^{c-Src} protein (see Kami et al., 2002), which has been implicated in several forms of human cancer (reviewed in Brickell, 1992). Thus, the compositions and methods of the presently claimed subject matter are applicable to cells that have elevated pp60^{c-Src} activity.

The elevation of pp60^{c-Src} or the loss of VHL function therefore allows the HIF-1 -selective conditionally replication competent adenovirus vectors to replicate in tumor cells (e.g. those derived from VHL-deficient clear cell renal carcinomas) in the absence of hypoxia. Under these circumstances, every tumor cell is targeted as HIF-1 is activated in every cell.

In one embodiment of the presently claimed subject matter, a method is provided for inhibiting the growth of a target tissue by co-infecting a cell in the target tissue with two different adenovirus vectors, one a conditionally replication competent vector comprising an adenovirus gene under the transcriptional regulation of an HRE, and the other a replication deficient adenovirus vector comprising a transgene. The use of a combination approach offers advantages in that a conditionally replication competent adenovirus has a capacity for a transgene of only about 2 kb (if the foreign promoter is small) to carry transgenes. Thus, there is a need to expand the capacity of an adenovirus vector to carry transgenes, which in many cases exceed 2 kb. With the use of a replication-deficient virus in conjunction with the conditionally replication competent virus, the ability to deliver transgenes can be significantly expanded. In the case of a first generation E1, E3 defective adenovirus vectors, the capacity will be about 8 kb. In the case of third generation gutless vectors, the capacity will reach approximately 37 kb. Construction of gutless vectors is described in Mitani et al., 1995; Fisher et al., 1996; Kochanek et al., 1996; Kumar-Singh & Chamberlain, 1996; Hardy et al., 1997; Parks & Graham, 1997; Morsy et al., 1998; PCT International Publication Nos. WO 98/54345; WO97/45550; and WO 96/33280; and U.S. Pat. No. 5,871,982.

V.A. Subjects

The subject treated in the presently claimed subject matter in its many embodiments is desirably a human subject, although it is to be understood that the principles of the presently claimed subject matter indicate that the presently claimed subject matter is effective with respect to invertebrate and to all vertebrate species, including mammals, which are intended to be included in the term “subject”. Moreover, a mammal is understood to include any mammalian species in which treatment or prevention of cancer is desirable, particularly agricultural and domestic mammalian species.

The methods of the presently claimed subject matter are particularly useful in the treatment of warm-blooded vertebrates. Thus, the presently claimed subject matter concerns mammals and birds.

More particularly provided is the treatment of mammals such as humans, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, i.e., poultry, such as turkeys, chickens, ducks, geese, guinea fowl, and the like, as they are also of economic importance to humans. Thus, contemplated is the treatment of livestock, including, but not limited to, domesticated swine (pigs and hogs), ruminants, horses, poultry, and the like.

V.B. Formulation

The adenovirus vectors of the presently claimed subject matter comprise in one embodiment a composition that includes a pharmaceutically acceptable carrier. Any suitable pharmaceutical formulation can be used to prepare the adenovirus vectors for administration to a subject.

For example, suitable formulations can include aqueous and non-aqueous sterile injection solutions which can contain anti-oxidants, buffers, bacteriostats, bactericidal antibiotics and solutes which render the formulation isotonic with the bodily fluids of the intended recipient; and aqueous and non-aqueous sterile suspensions which can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients are SDS, in one example in the range of 0.1 to 10 mg/ml, in another example about 2.0 mg/ml; and/or mannitol or another sugar, for example in the range of 10 to 100 mg/ml, in another example about 30 mg/ml; and/or phosphate-buffered saline (PBS).

It should be understood that in addition to the ingredients particularly mentioned above the formulations of this presently claimed subject matter can include other agents conventional in the art having regard to the type of formulation in question. Of the possible formulations, sterile pyrogen-free aqueous and non-aqueous solutions can be used.

The therapeutic regimens and pharmaceutical compositions of the presently claimed subject matter can be used with additional adjuvants or biological response modifiers including, but not limited to, the cytokines IFN-α, IFN-γ, IL2, IL4, IL6, TNF, or other cytokine affecting immune cells. In accordance with this aspect of the presently claimed subject matter, the disclosed adenovirus vector can be administered in combination therapy with one or more of these cytokines.

V.C. Administration

Suitable methods for administration of an adenovirus vector of the presently claimed subject matter include but are not limited to intravenous or intratumoral injection. Alternatively, an adenovirus vector can be deposited at a site in need of treatment in any other manner, for example by spraying a composition comprising an adenovirus vector within the pulmonary pathways. The particular mode of administering a therapeutic composition of the presently claimed subject matter depends on various factors, including the distribution and abundance of cells to be treated, the vector employed, additional tissue- or cell-targeting features of the vector, and mechanisms for metabolism or removal of the vector from its site of administration. For example, relatively superficial tumors can be injected intratumorally. By contrast, internal tumors can be treated by intravenous injection.

In one embodiment, the method of administration encompasses features for regionalized vector delivery or accumulation at the site in need of treatment. In one example, an adenovirus vector is delivered intratumorally. In another embodiment, selective delivery of a adenovirus vector to a tumor is accomplished by intravenous injection of the construct For delivery of adenovirus vectors to pulmonary pathways, adenovirus vectors of the presently claimed subject matter can be formulated as an aerosol or coarse spray. Methods for preparation and administration of aerosol or spray formulations can be found, for example, in Cipolla et al., 2000 and in U.S. Pat. Nos. 5,858,784; 6,013,638; 6,022,737; and 6,136,295.

V.D. Dose

An effective dose of an adenovirus vector composition of the presently claimed subject matter is administered to a subject in need thereof. A “therapeutically effective amount” is an amount of the therapeutic composition sufficient to produce a measurable response (e.g., a cytolytic response in a subject being treated). In one embodiment, an activity that inhibits tumor growth is measured. Actual dosage levels of active ingredients in the pharmaceutical compositions of this presently claimed subject matter can be varied so as to administer an amount of the active compound(s) that is effective to achieve the desired therapeutic response for a particular subject. The selected dosage level will depend upon the activity of the therapeutic composition, the route of administration, combination with other drugs or treatments, the severity of the condition being treated, and the condition and prior medical history of the subject being treated. However, it is within the skill of the art to start doses of the compound at levels lower than required to achieve the desired therapeutic effect and to gradually increase the dosage until the desired effect is achieved.

The potency of a therapeutic composition can vary, and therefore a “therapeutically effective” amount can vary. However, using the assay methods described herein below, one skilled in the art can readily assess the potency and efficacy of a candidate modulator of this presently claimed subject matter and adjust the therapeutic regimen accordingly.

After review of the disclosure herein of the presently claimed subject matter, one of ordinary skill in the art can tailor the dosages to an individual patient, taking into account the particular formulation, method of administration to be used with the composition, and tumor size. Further calculations of dose can consider patient height and weight, severity and stage of symptoms, and the presence of additional deleterious physical conditions. Such adjustments or variations, as well as evaluation of when and how to make such adjustments or variations, are well known to those of ordinary skill in the art of medicine.

For local administration of viral vectors, previous clinical studies have demonstrated that up to 10¹³ plaque forming units (pfu) of virus can be injected with minimal toxicity. In human patients, 1×10⁹-1×10¹³ pfu are routinely used (see Habib et al., 1999). To determine an appropriate dose within this range, preliminary treatments can begin with 1×10⁹ pfu, and the dose level can be escalated in the absence of dose-limiting toxicity. Toxicity can be assessed using criteria set forth by the National Cancer Institute and is reasonably defined as any grade 4 toxicity or any grade 3 toxicity persisting more than 1 week. Dose is also modified to maximize anti-tumor or anti-angiogenic activity. Representative criteria and methods for assessing anti-tumor and/or anti-angiogenic activity are described herein below. With replicative virus vectors, a dosage of about 1×10⁷ to 1×10⁸ pfu can be used in some instances.

Indeed, in one embodiment the presently claimed subject matter provides a method of selectively propagating an adenovirus in a target tissue, such as a tumor, another hypoxic tissue, or other tissue expressing HIF-1. An adenovirus construct as disclosed herein is packaged into adenovirus vectors and the prepared virus titer reaches at least 1×10⁶-1×10⁷ pfu/ml. The adenoviral construct is administered in the amount of 1.0 pfu/target cell. Thus, administration of a minimal level of adenoviral construct to thereby provide a therapeutic level upon propagation of the virus comprises an aspect of the presently claimed subject matter.

### EXAMPLES

The following Examples have been included to illustrate modes of the presently claimed subject matter. Certain aspects of the following Examples are described in terms of techniques and procedures found or contemplated by the present co-inventors to work well in the practice of the presently claimed subject matter. These Examples illustrate standard laboratory practices of the co-inventors. In light of the present disclosure and the general level of skill in the art, those of skill will appreciate that the following Examples are intended to be exemplary only and that numerous changes, modifications, and alterations can be employed without departing from the scope of the presently claimed subject matter.

### Example 1

### In Vitro Expression of EGFP in Cells Exposed to Hypoxia

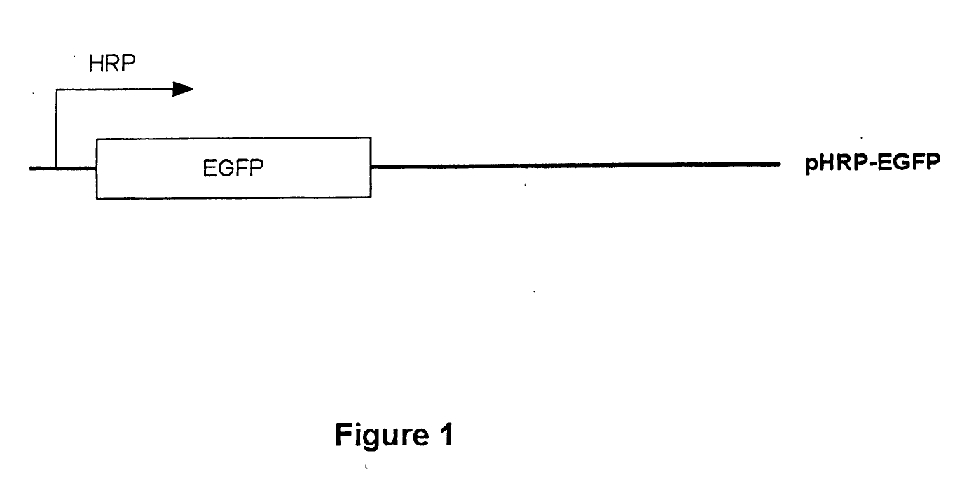

A promoter based on the HIF-1 binding elements in the VEGF promoter was constructed. The hypoxia responsive promoter (HRP) comprises 5 tandem copies of the HRE from the human VEGF promoter linked to the minimal promoter from cytomegalovirus (CMV). In order to test the activity of this promoter, a plasmid, depicted in FIG. 1, was constructed in which the HRP controlled the expression of the enhanced green fluorescence protein (EGFP) gene. The HRP-EGFP construct was used to establish stable sublines from two tumor cell lines: HCT116, a human colon carcinoma cell line; and 4T1, a murine mammary adenocarcinoma. Cells from stably transduced sublines exposed to hypoxic conditions (with oxygen tension at 0.5 to 1.5%), showed robust expression of EGFP 24 hours after incubation.

### Example 2

### HRP-Driven EGFP Expression in Subcutaneous Tumors

Tumors were established by injecting 10⁵-10⁶ cells into mice subcutaneously. The injected cells were 4T1 cells stably transduced with a construct (HRP-EGFP; see FIG. 1) comprising an artificial hypoxia responsive promoter controlling the expression of the EGFP gene. Tumors were allowed to grow to approximately 5-8 mm in diameter. Right before excising the tumor and sacrificing the mice, mice were injected with pimonidazole intraperitoneally. Pimonidazole staining is a standard method for identifying hypoxic regions within tumors (Raleigh et al., 1998). Frozen sections of the tumors were then stained with an anti-pimonidazole antibody and observed under a fluorescence microscope. The EGFP expression patterns from the same sections were also observed. Concordant patterns of EGFP expression and pimonidazole staining were observed for each section, confirming the suitability of the HRP-EGFP reporter in reporting hypoxic tumor regions.

### Example 3

### In Vitro Replication of Conditionally Replication Competent Adenovirus Vectors

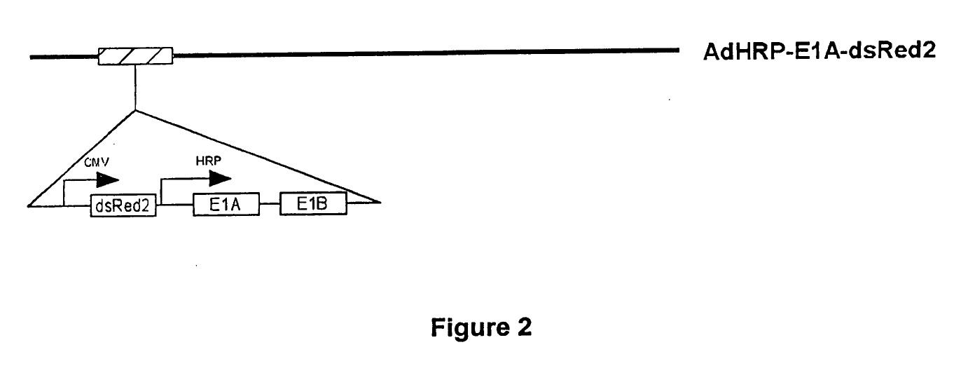

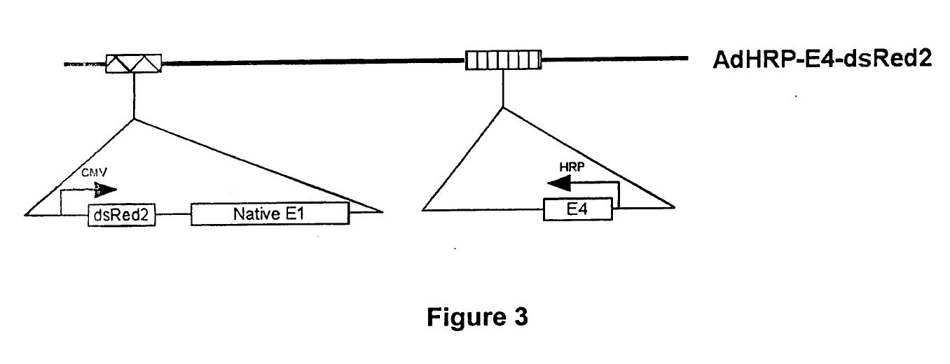

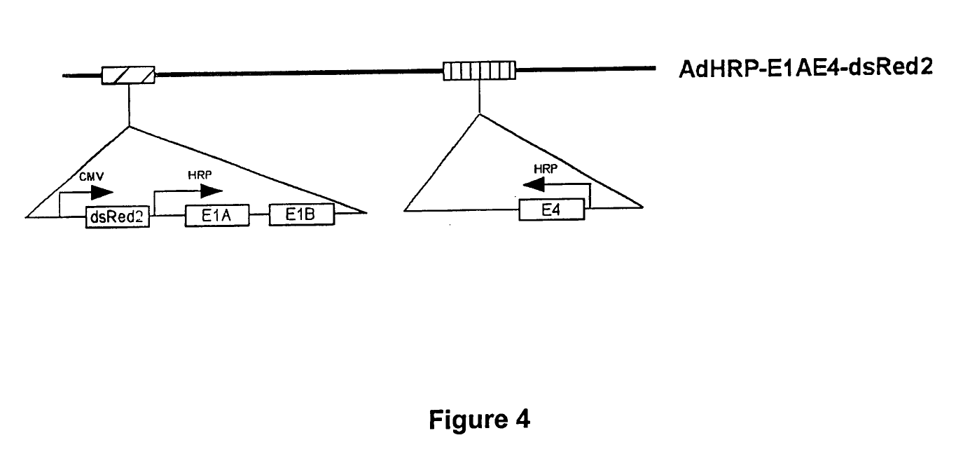

An adenovirus vector comprising the adenovirus E1A gene under the control of the HRP promoter was constructed (AdHRP-E1A-dsRed2; see FIG. 2). A reporter gene encoding a red fluorescent protein (dsRed2) was engineered into the vector to facilitate tracing of virus infection and replication. This vector was then tested in the HCT116 human colon carcinoma cell line. Hypoxia led to active replication of this virus vector. Fluorescence microscopy demonstrated significantly more virus replication and infection in the cells exposed to hypoxia. When measured by flow cytometry, the differential in dsRed2 expression was at least 100 fold, which was confirmed by plaque forming assays. Western blot analysis of E1A protein showed that E1A is expressed at a significant level only in cells that were subjected to hypoxic conditions.

### Example 4

### In Vivo Replication of Conditionally Replication Competent Vectors

HCT116 cells transduced with HRP-EGFP constructs (see FIG. 1) were used to establish tumors in nude mice. Mice bearing these tumors were then infected with an adenovirus vector (AdHRP-E1A-dsRed2; see FIG. 2) that carried a red fluorescent protein. The tumor cells expressed the EGFP protein under the control of the HRP while the virus vector encoded a red fluorescent marker, allowing comparison of the relative expression patterns of virus replication and tumor hypoxia.

Reporter-transduced HCT116 cells were injected subcutaneously into nude mice. Tumors grew up in 34 weeks to sizes of 8-10 mm in diameter. AdHRP-E1A-dsRed2 was injected intratumorally at a dosage of 1×10⁸ plaque forming units (pfu). The animals were sacrificed 3-10 days later and the tumors were excised and sectioned for analysis. The hypoxia responsive vector replicated with tremendous efficiency in hypoxic regions, leading to high-level expression of dsRed2. The expression of dsRed2 was concordant with that of the EGFP, indicating selectivity in hypoxic regions of the tumor. In addition, the hypoxia-responsive promoter demonstrated tremendous advantage over a non-replicative adenovirus vector Ad-CMV-dsRed2 gene under the control of a CMV promoter. Cells infected with the replication deficient dsRed2 virus showed low efficiency both in terms of infected tumor area and in terms of fluorescence intensity. These results demonstrated the significant advantages of the hypoxia-selective replication-competent adenovirus.

### Example 5

### In Vivo Tumor Growth Inhibition

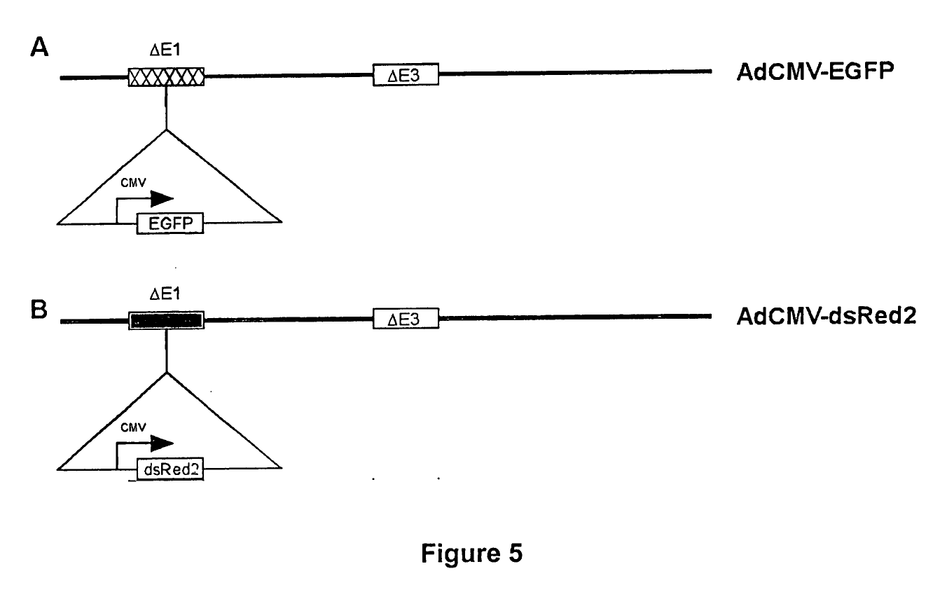

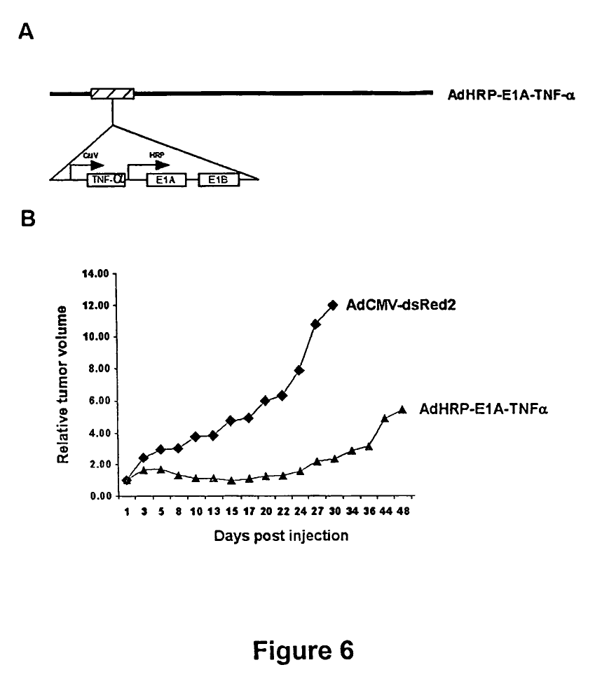

HCT116 (human colon cancer) cells were injected subcutaneously into nude mice at 3.0×10⁶ cell/mouse. When the tumors reach 5-10 mm in diameter, viral vectors were injected intratumorally. The control group (FIG. 6B, solid squares) was injected with AdCMV-dsRed2 (FIG. 5), while the treatment group (FIG. 6B, solid triangles) was injected with AdHRP-E1A-TNF-α (FIG. 6A), which was a conditionally replication competent adenovirus vector comprising the E1A gene operably linked to an HRP and further comprising a constitutively expressed TNF-α gene. 2.0×10⁹ pfu of the appropriate virus was injected intratumorally per tumor. Tumor volume was determined every 2-3 days. The relative volume was calculated by setting the volume of each tumor at day zero (i.e., the time point immediately preceding vector injection) at 1.0. As shown in FIG. 6B, tumors injected with the conditionally replication competent adenovirus vector grew considerably more slowly than the control.

### Example 6

### Replication of E1-Deficient AdCMV-EGFP in the Presence of a Conditionally Replication Competent Adenovirus Vector

The ability of a conditionally replication competent adenovirus vector to support the replication of a replication deficient adenovirus vector was tested. A replication deficient adenovirus vector, AdCMV-EGFP (see FIG. 5) was constructed that encoded a constitutively active EGFP gene. In this vector, the E1 and E3 genes are deleted and the EGFP gene (under the control of a constitutively active CMV promoter) is inserted into the E1 region of the virus. The conditionally replication competent adenovirus vector AdHRP-E1A-dsRed2 (see FIG. 2), which encodes a constitutively active dsRed protein and was described above, was used.

HCT116 colon cancer cells at 90% confluence were infected with each of the two vectors at a multiplicity of infection (MOI) of 0.5 for each virus. Five hours after infection, the cells were subjected to hypoxia (1% O₂ concentration) in a Bactron chamber for 24 hours. After the hypoxic incubation, the cells were further incubated under normoxic condition for 24 hours. Cells were then viewed using fluorescence microscopy for EGFP and dsRed expression. The vast majority of cells was positive for both fluorescent markers, or was negative for both. Very few cells were positive for only one marker or the other. The presence of cells positive for both fluorescent markers indicates that co-infection of cells with the conditionally replication competent vector allows the replication-defective adenovirus to replicate and efficiently express encoded genes.

### References

The references listed below as well as all references cited in the specification are incorporated herein by reference to the extent that they supplement, explain, provide a background for or teach methodology, techniques and/or compositions employed herein.
Adelman J P, Hayflick J S, Vasser M & Seeburg P H (1983) In Vitro Deletional Mutagenesis for Bacterial Production of the 20,000-Dalton Form of Human Pituitary Growth Hormone. DNA 2:183-193.
Altschul S F, Gish W, Miller W, Myers E W & Lipman D J (1990) Basic Local Alignment Search Tool. J Mol Biol 215:403-410.
Ausubel F M, Brent R, Kingston R E, Moore D D, Seidman J G, Smith J A & Struhl K, eds. (1992) Current Protocols in Molecular Biology. Wiley, New York.
Barton G J (1998) Protein Sequence Alignment Techniques. Acta Crystallogr D Biol Crystallogr 54:1139-1146.
Batzer M A, Carlton J E & Deininger P L (1991) Enhanced Evolutionary Pcr Using Oligonucleotides with Inosine at the 3′-Terminus. Nucleic Acids Res 19:5081.
Beroud C & Soussi T (1998) p53 Gene Mutation: Software and Database. Nucleic Acids Res 26:200-204.
Bischoff J R, Kim D H, Williams A, Heise C, Horn S, Muna M, Ng L, Nye J A, Sampson-Johannes A, Fattaey A & McCormick F (1996) An Adenovirus Mutant That Replicates Selectively in p53-Deficient Human Tumor Cells. Science 274:373-376.
Brickell P M (1992) The P60c-Src Family of Protein-Tyrosine Kinases: Structure, Regulation, and Function. Crit Rev Oncog 3:401-446.
Cipolla D C, Gonda I, Shak S, Kovesdi I, Crystal R & Sweeney T D (2000) Coarse Spray Delivery to a Localized Region of the Pulmonary Airways for Gene Therapy. Hum Gene Ther 11:361-371.
Clifford S C & Maher E R (2001) Von Hippel-Lindau Disease: Clinical and Molecular Perspectives. Adv Cancer Res 82:85-105.
Colman M S, Afshari C A & Barrett J C (2000) Regulation of p53 Stability and Activity in Response to Genotoxic Stress. Mutat Res 462:179-188.
Cubitt A B, Heim R, Adams S R, Boyd A E, Gross L A & Tsien R Y (1995) Understanding, Improving and Using Green Fluorescent Proteins. Trends Biochem Sci 20:448-455.
Dachs G U & Tozer G M (2000) Hypoxia Modulated Gene Expression: Angiogenesis, Metastasis and Therapeutic Exploitation. Eur J Cancer 36:1649-1660.
D'Andrea A, Rengaraju M, Valiante N M, Chehimi J, Kubin M, Aste M, Chan S H, Kobayashi M, Young D, Nickbarg E et al. (1992) Production of Natural Killer Cell Stimulatory Factor (Interleukin 12) by Peripheral Blood Mononuclear Cells. J Exp Med 176:1387-1398.
Deshmukh P, Glick R P, Lichtor T, Moser R & Cohen E P (2001) Immunogene Therapy with Interleukin-2-Secreting Fibroblasts for Intracerebrally Metastasizing Breast Cancer in Mice. J Neurosurg 94:287-292.
Dias S, Thomas H & Balkwill F (1998) Multiple Molecular and Cellular Changes Associated with Tumour Stasis and Regression During II-12 Therapy of a Murine Breast Cancer Model. Int J Cancer 75:151-157.
Dix B R, Edwards S J & Braithwaite A W (2001) Does the Antitumor Adenovirus Onyx-015/DI1520 Selectively Target Cells Defective in the p53 Pathway? J Virol 75:5443-5447.
European Patent No. 0 439 095
Firth J, Ebert B, Pugh C & Ratcliffe P (1994) Oxygen-Regulated Control Elements in the Phosphoglycerate Kinase 1 and Lactate Dehydrogenase A Genes: Similarities with the Erythropoietin 3′ Enhancer. PNAS 91:6496-6500.
Fisher K J, Choi H, Burda J, Chen S J & Wilson J M (1996) Recombinant Adenovirus Deleted of All Viral Genes for Gene Therapy of Cystic Fibrosis. Virology 217:11-22.
Forsythe J, Jiang B, Iyer N, Agani F, Leung S K, R D & Semenza G (1996) Activation of Vascular Endothelial Growth Factor Gene Transcription by Hypoxia inducible Factor 1. Mol Cell Biol 16:4604-4613.
Frankel A E, Powell B L, Vallera D A & Neville D M, Jr. (2001) Chimeric Fusion Proteins-Diphtheria Toxin-Based. Curr Opin Investig Drugs 2:1294-1301.
Freeman S M, Whartenby K A, Freeman J L, Abboud C N & Marrogi A J (1996) In Situ Use of Suicide Genes for Cancer Therapy. Semin Oncol 23:31-45.
Galanis E, Vile R & Russell S J (2001) Delivery Systems Intended for in Vivo Gene Therapy of Cancer Targeting and Replication Competent Viral Vectors. Crit Rev Oncol Hematol 38:177-192.
Gately M K, Warrier R R, Honasoge S. Carvajal D M, Faherty D A, Connaughton S E, Anderson T D, Sarmiento U, Hubbard B R & Murphy M (1994) Administration of Recombinant II-12 to Normal Mice Enhances Cytolytic Lymphocyte Activity and Induces Production of Ifn-Gamma in Vivo. Int Immunol 6:157-167.
Glover D M & Hames B D (1995) DNA Cloning : A Practical Approach, 2nd ed. IRL Press at Oxford University Press, Oxford; N.Y.
Goodrum F D & Ornelles D A (1998) p53 Status Does Not Determine Outcome of E1B 55-Kilodalton Mutant Adenovirus Lytic Infection. J Virol 72:9479-9490.
Gore M (1996) The Role of lnterleukin-2 in Cancer Therapy. Cancer Biother Radiopharm 11:281-283.
Greenberg N M, DeMayo F J, Sheppard P C, Barrios R, Lebovitz R, Finegold M, Angelopoulou R, Dodd J G, Duckworth M L, Rosen J M et al. (1994) The Rat Probasin Gene Promoter Directs Hormonally and Developmentally Regulated Expression of a Heterologous Gene Specifically to the Prostate in Transgenic Mice. Mol Endocrinol 8:230-239.
Habib N A, Hodgson H J, Lemoine N & Pignatelli M (1999) A Phase I/Ii Study of Hepatic Artery Infusion with wtp53-CMV-Ad in Metastatic Malignant Liver Tumours. Hum Gene Ther 10:2019-2034.
Hardy S, Kitamura M, Harris-Stansil T. Dai Y & Phipps M L (1997) Construction of Adenovirus Vectors through Cre-Lox Recombination. J Virol 71:1842-1849.
Haviv Y S & Curiel D T (2001) Conditional Gene Targeting for Cancer Gene Therapy. Adv Drug Deliv Rev 53:135-154.
Henikoff J G, Pietrokovski S, McCallum C M & Henikoff S (2000) Blocks-Based Methods for Detecting Protein Homology. Electrophoresis 21:1700-1706.
Henikoff S & Henikoff J G (1992) Amino Acid Substitution Matrices from Protein Blocks. Proc Natl Acad Sci USA 89:10915-10919.
Henikoff S & Henikoff J G (2000) Amino Acid Substitution Matrices. Adv Protein Chem 54:73-97.
Hickman E S, Moroni M C & Helin K (2002) The Role of p53 and pRB in Apoptosis and Cancer. Curr Opin Genet Dev 12:60-66.
Horiguchi Y, Larchian W A, Kaplinsky R, Fair W R & Heston W D (2000) Intravesical Liposome-Mediated Interleukin-2 Gene Therapy in Orthotopic Murine Bladder Cancer Model. Gene Ther 7:844-851.
Hsieh C S, Macatonia S E, Tripp C S, Wolf S F, O'Garra A & Murphy K M (1993) Development of Th1 Cd4+ T Cells through II-12 Produced by Listeria-Induced Macrophages. Science 260:547-549.
Huang C C, Novak W R, Babbitt P C, Jewett A I, Ferrin T E & Klein T E (2000) Integrated Tools for Structural and Sequence Alignment and Analysis. Pac Symp Biocomput:230-241.
Idriss H T & Naismith J H (2000) TNFα and the TNF Receptor Superfamily: Structure-Function Relationship(S). Microsc Res Tech 50:184-195.
Ivan M & Kaelin W G, Jr. (2001) The Von Hippel-Lindau Tumor Suppressor Protein. Curr Opin Genet Dev 11:27-34.
Karlin S & Altschul S F (1993) Applications and Statistics for Multiple High-Scoring Segments in Molecular Sequences. Proc Natl Acad Sci USA 90:5873-5877.
Karni R, Dor Y, Keshet E, Meyuhas O & Levitzki A (2002) Activated Pp60c-Src Leads to Elevated Hif-1 Alpha Expression under Normoxia. J Biol Chem:M206141200.
Kirk C J & Mule J J (2000) Gene-Modified Dendritic Cells for Use in Tumor Vaccines. Hum Gene Ther 11:797-806.
Klohs W D, Fry D W & Kraker A J (1997) Inhibitors of Tyrosine Kinase. Curr Opin Oncol 9:562-568.
Kobayashi M, Fitz L, Ryan M, Hewick R M, Clark S C, Chan S, Loudon R, Sherman F, Perussia B & Trinchieri G (1989) Identification and Purification of Natural Killer Cell Stimulatory Factor (Nksf), a Cytokine with Multiple Biologic Effects on Human Lymphocytes. J Exp Med 170:827-845.
Kochanek S, Clemens P R, Mitani K, Chen H H, Chan S & Caskey C T (1996) A New Adenoviral Vector: Replacement of All Viral Coding Sequences with 28 Kb of DNA Independently Expressing Both Full-Length Dystrophin and Beta-Galactosidase. Proc Natl Acad Sci USA 93:5731-5736.
Kumar-Singh R & Chamberlain J S (1996) Encapsidated Adenovirus Minichromosomes Allow Delivery and Expression of a 14 Kb Dystrophin Cdna to Muscle Cells. Hum Mol Genet 5:913-921.
Kurihara T, Brough D E, Kovesdi I & Kufe D W (2000) Selectivity of a Replication competent Adenovirus for Human Breast Carcinoma Cells Expressing the MUC1 Antigen. J Clin Invest 106:763-771.
Kyte J & Doolittle R F (1982) A Simple Method for Displaying the Hydropathic Character of a Protein. J Mol Biol 157:105-132.
Larchian W A, Horiguchi Y, Nair S K, Fair W R, Heston W D & Gilboa E (2000) Effectiveness of Combined Interleukin 2 and B7.1 Vaccination Strategy Is Dependent on the Sequence and Order: A Liposome-Mediated Gene Therapy Treatment for Bladder Cancer. Clin Cancer Res 6:2913-2920.
Lee S E, Jin R J, Lee S G, Yoon S J, Park M S, Heo D S & Choi H (2000) Development of a New Plasmid Vector with PSA-Promoter and Enhancer Expressing Tissue-Specificity in Prostate Carcinoma Cell Lines. Anticancer Res 20:417-422.
Lin P, Sankar S, Shan S, Dewhirst M W, Polverini P J, Quinn T Q & Peters K G (1998) Inhibition of Tumor Growth by Targeting Tumor Endothelium Using a Soluble Vascular Endothelial Growth Factor Receptor. Cell Growth Differ 9:49-58.
Lindegaard J C, Overgaard J, Bentzen S M & Pedersen D (1996) Is There a Radiobiologic Basis for Improving the Treatment of Advanced Stage Cervical Cancer? J Natl Cancer Inst Monogr 21:105-112.
Liu Y, Cox S. Morita T & Kourembanas S (1995) Hypoxia Regulates Vascular Endothelial Growth Factor Gene Expression in Endothelial Cells. Identification of a 5′ Enhancer. Circ Res 77.
Luna M C, Ferrario A, Wong S, Fisher A M & Gomer C J (2000) Photodynamic Therapy-Mediated Oxidative Stress as a Molecular Switch for the Temporal Expression of Genes Ligated to the Human Heat Shock Promoter. Cancer Res 60:1637-1644.
Mackensen A, Lindemann A & Mertelsmann R (1997) Immunostimulatory Cytokines in Somatic Cells and Gene Therapy of Cancer. Cytokine Growth Factor Rev 8:119-128.
Majewski S, Marczak M, Szmurlo A, Jablonska S & Bollag W (1996) Interleukin-12 Inhibits Angiogenesis Induced by Human Tumor Cell Lines in Vivo. J Invest Dermatol 106:1114-1118.
Manetti R, Parronchi P, Giudizi M G, Piccinni M P, Maggi E, Trinchieri G & Romagnani S (1993) Natural Killer Cell Stimulatory Factor (Interleukin 12 [II-12]) Induces T Helper Type 1 (Th1)-Specific Immune Responses and Inhibits the Development of II-4-Producing Th Cells. J Exp Med 177:1199-1204.
Margolin K A (2000) Interleukin-2 in the Treatment of Renal Cancer. Semin Oncol 27:194-203.
Maxwell P H, Pugh C W & Ratcliffe P J (2001) Activation of the Hif Pathway in Cancer. Curr Opin Genet Dev 11:293-299.
Mitani K, Graham F L, Caskey C T & Kochanek S (1995) Rescue, Propagation, and Partial Purification of a Helper Virus-Dependent Adenovirus Vector. Proc Natl Acad Sci USA 92:3854-3858.
Morsy M A, Gu M, Motzel S, Zhao J, Lin J, Su Q, Allen H, Franlin L, Parks R J, Graham F L, Kochanek S, Bett A J & Caskey C T (1998) An Adenoviral Vector Deleted for All Viral Coding Sequences Results in Enhanced Safety and Extended Expression of a Leptin Transgene. Proc Natl Acad Sci USA 95:7866-7871.
Narvaiza I, Mazzolini G, Barajas M, Duarte M, Zaratiegui M, Qian C, Melero I & Prieto J (2000) Intratumoral Coinjection of Two Adenoviruses, One Encoding the Chemokine Ifn-Gamma-Inducible Protein-10 and Another Encoding II-12, Results in Marked Antitumoral Synergy. J Immunol 164:3112-3122.
Needleman S B & Wunsch C D (1970) A General Method Applicable to the Search for Similarities in the Amino Acid Sequence of Two Proteins. J Mol Biol 48:443-453.
Nomura T & Hasegawa H (2000) Chemokines and Anti-Cancer Immunotherapy: Anti-Tumor Effect of Ebil-Ligand Chemokine (Elc) and Secondary Lymphoid Tissue Chemokine (Sic). Anticancer Res 20:4073-4080.
O'Byrne K J, Daigleish A G, Browning M J, Steward W P & Harris A L (2000) The Relationship between Angiogenesis and the Immune Response in Carcinogenesis and the Progression of Malignant Disease. Eur J Cancer 36:151-169.
Ohtsuka E, Matsuki S, Ikehara M, Takahashi Y & Matsubara K (1985) An Alternative Approach to Deoxyoligonucleotides as Hybridization Probes by Insertion of Deoxyinosine at Ambiguous Codon Positions. J Biol Chem 260:2605-2608.
Overwijk W W, Theoret M R & Restifo N P (2000) The Future of Interleukin-2: Enhancing Therapeutic Anticancer Vaccines. Cancer J Sci Am 6:S76-80.
Parks R J & Graham F L (1997) A Helper-Dependent System for Adenovirus Vector Production Helps Define a Lower Limit for Efficient DNA Packaging. J Virol 71:3293-3298
Patterson L H (2002) Bioreductively Activated Antitumor N-Oxides: The Case of AQ4N, a Unique Approach to Hypoxia-Activated Cancer Chemotherapy. Drug Metab Rev 34:581-592.
PCT International Publication No. WO 96/33280
PCT International Publication No. WO 97/45550
PCT International Publication No. WO 97/47763
PCT International Publication No. WO 98/54345
Pearson W R & Lipman D J (1988) Improved Tools for Biological Sequence Comparison. Proc Natl Acad Sci USA 85:2444-2448.
Porter W, Wang F, Duan R, Qin C, Castro-Rivera E, Kim K & Safe S (2001) Transcriptional Activation of Heat Shock Protein 27 Gene Expression by 17β-Estradiol and Modulation by Antiestrogens and Aryl Hydrocarbon Receptor Agonists. J Mol Endocrinol 26:31-42.
Pugh C W, Tan C C, Jones R W & Ratcliffe P J (1991) Functional Analysis of an Oxygen-Regulated Transcriptional Enhancer Lying 3′ to the Mouse Erythropoietin Gene. Proc Natl Acad Sci USA 88:10553-10557.
Putzer B M, Hitt M, Muller W J, Emtage P, Gauldie J & Graham F L (1997) Interleukin 12 and B7-1 Costimulatory Molecule Expressed by an Adenovirus Vector Act Synergistically to Facilitate Tumor Regression. Proc Natl Acad Sci USA 94:10889-10894.
Raleigh J A, Calkins-Adams D P, Rinker L H, Ballenger C A, Weissler M C, Fowler W C, Jr., Novotny D B & Varia M A (1998) Hypoxia and Vascular Endothelial Growth Factor Expression in Human Squamous Cell Carcicomas Using Pimonidazole as a Hypoxia Marker. Cancer Res 58:3765-3768.
Ries S & Kom W M (2002) Onyx-015: Mechanisms of Action and Clinical Potential of a Replication-Selective Adenovirus. Br J Cancer 86:5-11.
Robertson M J, Soiffer R J, Wolf S F, Manley T J, Donahue C, Young D, Herrmann S H & Ritz J (1992) Response of Human Natural Killer (Nk) Cells to Nk Cell Stimulatory Factor (Nksf): Cytolytic Activity and Proliferation of Nk Cells Are Differentially Regulated by Nksf. J Exp Med 175:779-788.
Rossolini G M, Cresti S, Ingianni A, Cattani P, Riccio M L & Satta G (1994) Use of Deoxyinosine-Containing Primers Vs Degenerate Primers for Polymerase Chain Reaction Based on Ambiguous Sequence Information. Mol Cell Probes 8:91-98.
Rothmann T, Hengstermann A, Whitaker N J, Scheffner M & zur Hausen H (1998) Replication of Onyx-015, a Potential Anticancer Adenovirus, Is Independent of p53 Status in Tumor Cells. J Virol 72:9470-9478.
Sadekova S, Lehnert S & Chow T Y (1997) Induction of Pbp74/Mortalin/Grp75, a Member of the hsp70 Family, by Low Doses of Ionizing Radiation: A Possible Role in Induced Radioresistance. Int J Radiat Biol 72:653-660.
Sambrook J & Russell D W (2001) Molecular Cloning: A Laboratory Manual, 3rd ed. Cold Spring Harbor Laboratory Press, Cold Spring Harbor, N.Y.
Saqi M A, Wild D L & Hartshom M J (1999) Protein Analyst-a Distributed Object Environment for Protein Sequence and Structure Analysis. Bioinfonnatics 15:521-522.
Scharfmann R, Axelrod J H & Verma I M (1991) Long-Term in Vivo Expression of Retrovirus-Mediated Gene Transfer in Mouse Fibroblast Implants. Proc Natl Acad Sci USA 88:4626-4630.
Semenza G L, Nejfelt M K, Chi S M & Antonarakis S E (1991) Hypoxia inducible Nuclear Factors Bind to an Enhancer Element Located 3′ to the Human Erythropoietin Gene. Proc Natl Acad Sci USA 88:5680-5684.
Semenza G L, Roth P H, Fang H M & Wang G L (1994) Transcriptional Regulation of Genes Encoding Glycolytic Enzymes by Hypoxia inducible Factor 1. J Biol Chem 269:23757-23763.
Shen Y & White E (2001) p53-Dependent Apoptosis Pathways. Adv Cancer Res 82:55-84.
Shibuya M (2001) Structure and Dual Function of Vascular Endothelial Growth Factor Receptor-1 (Flt-1). Int J Biochem Cell Biol 33:409-420
Silhavy T J, Berman M L, Enquist L W & Cold Spring Harbor Laboratory. (1984) Experiments with Gene Fusions. Cold Spring Harbor Laboratory, Cold Spring Harbor, N.Y.
Sinkovics J G & Horvath J C (2000) Vaccination against Human Cancers (Review). Int J Oncol 16:81-96.
Smith T F & Waterman M (1981) Comparison of Biosequences. Adv Appl Math 2:482-489.
Srivastava R K (2001) Trail/Apo-2I: Mechanisms and Clinical Applications in Cancer. Neoplasia 3:535-546.
Suit H (1996) Assessment of the Impact of Local Control on Clinical Outcome. Front Radiat Ther Oncol 29:17-23.
U.S. Pat. No. 4,554,101
U.S. Pat. No. 5,858,784
U.S. Pat. No. 5,871,982
U.S. Pat. No. 6,013,638
U.S. Pat. No. 6,022,737
U.S. Pat. No. 6,136,295
Valter M M, Hugel A, Huang H J, Cavenee W K, Wiestler O D, Pietsch T & Wemert N (1999) Expression of the Ets-1 Transcription Factor in Human Astrocytomas Is Associated with Fms-Like Tyrosine Kinase-1 (Flt-1)/Vascular Endothelial Growth Factor Receptor-1 Synthesis and Neoangiogenesis. Cancer Res 59:5608-5614.
Voest E E, Kenyon B M, O'Reilly M S, Truitt G, D'Amato R J & Folkman J (1995) Inhibition of Angiogenesis in vivo by Interleukin 12. J Natl Cancer Inst 87:581-586.
Vose J M & Armitage J O (1995) Clinical Applications of Hematopoietic Growth Factors. J Clin Oncol 13:1023-1035.
Walther W & Stein U (1999) Therapeutic Genes for Cancer Gene Therapy. Mol Biotechnol 13:21-28.
Williams R S, Thomas J A, Fina M, German Z & Benjamin I J (1993) Human Heat Shock Protein 70 (Hsp70) Protects Murine Cells from Injury During Metabolic Stress. J Clin Invest 92:503-508.
Wolf S F, Temple P A, Kobayashi M, Young D, Dicig M, Lowe L, Dzialo R, Fitz L, Ferenz C, Hewick R M & et al. (1991) Cloning of Cdna for Natural Killer Cell Stimulatory Factor, a Heterodimeric Cytokine with Multiple Biologic Effects on T and Natural Killer Cells. J Immunol 146:3074-3081.
Yao L, Sgadari C, Furuke K, Bloom E T, Teruya-Feldstein J & Tosato G (1999) Contribution of Natural Killer Cells to Inhibition of Angiogenesis by Interleukin-12. Blood 93:1612-1621.
Yazawa K, Fisher W E & Brunicardi F C (2002) Current Progress in Suicide Gene Therapy for Cancer. World J Surg 26:783-789.
Yu D C, Chen Y, Seng M, Dilley J & Henderson D R (1999) The Addition of Adenovirus Type 5 Region E3 Enables Calydon Virus 787 to Eliminate Distant Prostate Tumor Xenografts. Cancer Res 59:4200-4203.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. An adenovirus vector comprising an adenovirus gene under the transcriptional control of a transcriptional regulatory element (TRE) comprising a minimal promoter and a hypoxia responsive element (HRE).

2. The adenovirus vector of claim 1, wherein the adenovirus gene is selected from the group consisting of an E1A gene, an E1B gene, an E2A gene, an E2B gene, and an E4 gene.

3. The adenovirus vector of claim 1, further comprising a second adenovirus gene under the transcriptional control of the TRE.

4. The adenovirus vector of claim 1, wherein the minimal promoter is selected from the group consisting of the cytomegalovirus (CMV) minimal promoter, the human β-actin minimal promoter, the human EF2 minimal promoter, and the adenovirus E1B minimal promoter.

5. The adenovirus vector of claim 4, wherein the CMV minimal promoter comprises SEQ ID NO: 1.

6. The adenovirus vector of claim 1, wherein the HRE is derived from the human vascular endothelial growth factor (VEGF) promoter.

7. The adenovirus vector of claim 6, wherein the HRE comprises SEQ ID NO: 2.

8. The adenovirus vector of claim 7, wherein the HRE comprises five tandem copies of SEQ ID NO: 2.

9. The adenovirus vector of claim 1, further comprising a transgene.

10. The adenovirus vector of claim 9, wherein the transgene is a second adenovirus gene.

11. The adenovirus vector of claim 9, wherein the transgene encodes an immunostimulatory molecule.

12. The adenovirus vector of claim 11, wherein the immunostimulatory molecule is selected from the group consisting of IL2 and IL12.

13. The adenovirus vector of claim 9, wherein the transgene is a suicide gene.

14. The adenovirus vector of claim 13, wherein the suicide gene is selected from the group consisting of a TNF-α gene, a Trail gene, a Bax gene, an HSV-tk gene, a cytosine deaminase gene, a p450 gene, and a diphtheria toxin gene, an s-Flt1 gene, and an ex-Flk1 gene.

15. A composition comprising the adenovirus vector of claim 1.

16. The composition of claim 15, further comprising a pharmaceutically acceptable carrier.

17. A method for suppressing tumor growth, the method comprising contacting a hypoxic cell in a tumor with the adenovirus vector of claim 1, whereby the vector enters the cell and inhibits tumor growth.

18. The method of claim 17, wherein the contacting results from administering the adenovirus vector via intratumoral injection.

19. The method of claim 17, wherein the contacting results from administering the adenovirus vector by intravenous injection.

20. The method of claim 17, further comprising exposing the tumor to a therapeutically effective amount of a second treatment, the second treatment chosen from the group consisting of ionizing radiation, chemotherapy, and photodynamic therapy.

21. A host cell comprising the adenovirus vector of claim 1.

22. A method for conferring selective cytotoxicity on a target cell, the method comprising contacting a cell that allows an HRE to function with the adenovirus of claim 1, whereby the adenovirus vector enters the cell.

23. A method for selectively propagating an adenovirus in target tissue expressing HIF-1, the method comprising contacting the target tissue with an adenovirus according to claim 1, whereby the adenovirus is propagated to a titer of at least 1.0×10⁷ pfu/ml.

24. A method of inhibiting growth of a target tissue, the method comprising:
(a) contacting a hypoxic cell in a target tissue with a first adenovirus vector, whereby the first adenovirus vector enters the cell; and
(b) contacting the hypoxic cell with a replication deficient adenovirus vector, whereby the replication deficient adenovirus vector enters the cell.

25. The method of claim 24, wherein the target tissue is a tumor.

26. The method of claim 24, wherein the first adenovirus vector comprises an adenovirus gene under the transcriptional control of a TRE comprising an HRE.

27. The method of claim 24, wherein the replication deficient adenovirus vector comprises a second gene.

28. The method of claim 27, wherein the replication deficient adenovirus vector comprises a second gene under the transcriptional control of a constitutive promoter.

29. The method of claim 27, wherein the replication deficient adenovirus vector comprises a second gene under the transcriptional control of a TRE comprising an HRE.

30. The method of claim 24, wherein:
(a) the first adenovirus vector comprises at least two essential adenovirus genes under the transcriptional control of a TRE comprising an HRE; and,
(b) the replication deficient adenovirus vector is deficient in at least two of the essential adenovirus genes under the transcriptional control control of a TRE comprising an HRE in the first adenovirus vector.

31. The method of claim 30, wherein the two essential adenovirus genes are each selected from the group consisting of an E1A gene, an E1B gene, an E2A gene, an E2B gene, and an E4 gene.

32. The method of claim 27, wherein the second gene is a suicide gene.

33. The method of claim 32, wherein the suicide gene is chosen from the group consisting of a TNF-α gene, a Trail gene, a Bax gene, an HSV-tk gene, a cytosine deaminase gene, a p450 gene, and a diphtheria toxin gene, an s-Flt1 gene, and an ex-Flk1 gene.

34. The method of claim 27, wherein the second gene encodes an immunostimulatory molecule.

35. The method of claim 32, wherein the immunostimulatory molecule is selected from the group consisting of IL2 and IL12.

36. The method of claim 24, further comprising exposing the target tissue to a therapeutically effective amount of a second treatment, the second treatment chosen from the group consisting of ionizing radiation, chemotherapy, and photodynamic therapy.


SEQUENCE LISTING (2 sequences)

SEQ ID NO: 1 - DNA, 60 bp, Cytomegalovirus
gatctgacggttcactaaacgagctctgcttatatagacctcccaccgtacacgcctacc

SEQ ID NO: 2 - DNA, 35 bp, Homo sapiens
ccacagtgcatacgtgggctccaacaggtcctctt